(19) United States
(12) Patent Application Publication
(10) Pub. No.: US 2024/0193777 A1
(43) Pub. Date: Jun. 13, 2024

(54) MEDICAL IMAGE PROCESSING DEVICE, MEDICAL IMAGE PROCESSING METHOD, AND PROGRAM

(71) Applicant: FUJIFILM Corporation, Tokyo (JP)

(72) Inventor: Keita OTANI, Tokyo (JP)

(73) Assignee: FUJIFILM Corporation, Tokyo (JP)

(21) Appl. No.: 18/587,962

(22) Filed: Feb. 27, 2024

Related U.S. Application Data
(63) Continuation of application No. PCT/JP2022/025286, filed on Jun. 24, 2022.

(30) Foreign Application Priority Data
Aug. 31, 2021 (JP) 2021-141456

(51) Int. Cl. G06T 7/00; A61B 6/00; G16H 30/20

(52) U.S. Cl. CPC G06T 7/0012; A61B 6/481; G16H 30/20; G06T 2207/20081; G06T 2207/20084

(57) ABSTRACT

Provided are a medical image processing device, a medical image processing method, and a program that can estimate temporal information in an image to be processed even in a case where it is difficult to use information attached to the image to be processed. A medical image processing device includes one or more processors and one or more memories that store a program to be executed by the one or more processors. The one or more processors execute commands of the program to receive an input of an image generated by performing contrast imaging (1002) and to estimate an elapsed period from start of injection of a contrast agent in the image on the basis of image analysis of the image (1004).

### CROSS-REFERENCE TO RELATED APPLICATIONS

The present application is a Continuation of PCT International Application No. PCT/JP2022/025286 filed on Jun. 24, 2022 claiming priority under 35 U.S.C § 119(a) to Japanese Patent Application No. 2021-141456 filed on Aug. 31, 2021. Each of the above applications is hereby expressly incorporated by reference, in its entirety, into the present application.

### BACKGROUND OF THE INVENTION

### 1. Field of the Invention

The present invention relates to a medical image processing device, a medical image processing method, and a program.

### 2. Description of the Related Art

An imaging method called dynamic contrast-enhanced CT, which is a combination of angiography using a contrast agent and X-ray CT, is known. After the contrast agent is injected into a subject, the subject is imaged at different time points to acquire a plurality of three-dimensional contrast images. In addition, the dynamic contrast-enhanced CT may be referred to as dynamic CT, contrast-enhanced dynamic CT, or the like. Further, CT is an abbreviation of Computed Tomography.

The appearance of a contrast image acquired from imaging using dynamic contrast-enhanced CT significantly differs depending on a contrast state such as a contrast time phase. Therefore, in a process, such as blood vessel extraction, that depends on the contrast, it is necessary to accurately understand the contrast state.

JP2011-136030A discloses an image determination device that automatically determines whether an image is a contrast image or a non-contrast image. The device disclosed in JP2011-136030A detects a region of a first part which is not affected by a contrast agent from acquired image data, specifies a region of a second part which has a predetermined relative positional relationship with the first part and is affected by the contrast agent, and determines whether or not the image data has been obtained by contrast imaging according to whether or not a CT value of the second region is equal to or greater than a predetermined value.

JP5357818B discloses an image processing device that extracts a specific region from a three-dimensional medical image. The device disclosed in JP5357818B acquires time information related to an imaging time point of a medical image from input volume data of the medical image and estimates a contrast time phase of the medical image on the basis of the acquired time information. In a case where the device acquires the time information related to the imaging time point of the medical image from the input volume data of the medical image, the device acquires the time information including a contrast agent injection time point and the imaging time point from a tag that is called a DICOM header attached to the medical image. In addition, DICOM is an abbreviation of Digital Imaging and Communication in Medicine.

Michal Sofka, Dijia Wu, Michael Suhling, David Liu, Christian Tietjen, Grzegorz Soza, S. Kevin Zhou, et al., “Automatic Contrast Phase Estimation in CT Volumes”, MICCAI 2011, Part III, LNCS 6893, p. 166-174 discloses an automatic algorithm of phase labeling that depends on a change in the intensity of an anatomical region due to propagation of a contrast agent. A method disclosed in Michal Sofka, Dijia Wu, Michael Suhling, David Liu, Christian Tietjen, Grzegorz Soza, S. Kevin Zhou, et al., “Automatic Contrast Phase Estimation in CT Volumes”, MICCAI 2011, Part III, LNCS 6893, p. 166-174 detects a specific region and determines a contrast time phase of the detected region on the basis of a histogram of the detected region. Specifically, the method disclosed in Michal Sofka, Dijia Wu, Michael Suhling, David Liu, Christian Tietjen, Grzegorz Soza, S. Kevin Zhou, et al., “Automatic Contrast Phase Estimation in CT Volumes”, MICCAI 2011, Part III, LNCS 6893, p. 166-174 automatically discriminates whether the phase is a pre-contrast phase, an arterial phase, a portal phase, or an equilibrium phase from a contrast image of the liver.

### SUMMARY OF THE INVENTION

However, there may be a case where the information related to the imaging time point and the like is not included in the DICOM tag and a case where the information related to the imaging time point and the like included in the DICOM tag is incorrect. In this case, it is difficult to acquire accurate information related to the imaging time point and the like, and a method other than the method based on the information included in the DICOM tag is required.

A system disclosed in JP2011-136030A determines whether the acquired image data is a contrast image or a non-contrast image, and it is difficult to estimate temporal information in the contrast image.

The device disclosed in JP5357818B acquires the information related to the imaging time point of the medical image and the like from the DICOM header. In a case where the information related to the imaging time point and the like is not included in the DICOM header, it is difficult to acquire the information related to the imaging time point of the medical image and the like. Further, in a case where the information related to the imaging time point included in the DICOM header is incorrect, it is not possible to use the acquired information related to the imaging time point.

In the method disclosed in Michal Sofka, Dijia Wu, Michael Suhling, David Liu, Christian Tietjen, Grzegorz Soza, S. Kevin Zhou, et al., “Automatic Contrast Phase Estimation in CT Volumes”, MICCAI 2011, Part III, LNCS 6893, p. 166-174, in a case where a classifier in which a learning-based identification algorithm is used is generated, it is necessary to manually generate teaching data for the classifier. In a case where image data for each contrast state is prepared, it is difficult to define the contrast state for each image data item. Furthermore, there may be room for arbitrariness in the definition of the contrast state.

In addition, since the contrast state may change depending on an examination protocol, it is difficult to add image data, in which a different examination protocol has been used, to learning data. Further, since the definition of the contrast state differs depending on the organ, the contrast state is fixed for each organ, and it is difficult to change the contrast state.

The present invention has been made in view of these circumstances, and an object of the present invention is to provide a medical image processing device, a medical image processing method, and a program that can estimate temporal information in an image to be processed even in a case where it is difficult to use information attached to the image to be processed.

According to the present disclosure, there is provided a medical image processing device comprising: one or more processors; and one or more memories that store a program to be executed by the one or more processors. The one or more processors execute commands of the program to receive an input of an image generated by performing contrast imaging and to estimate an elapsed period from start of injection of a contrast agent in the image on the basis of image analysis of the image.

According to the medical image processing device of the present disclosure, the elapsed period from the start of the injection of the contrast agent in the image is estimated on the basis of the image analysis of the input image. Therefore, even in a case where it is difficult to use information, such as an imaging start time point, that is attached to the image, it is possible to estimate the elapsed period from the start of the injection of the contrast agent in the image.

The image can include the meaning of image data which is a signal indicating the image.

The term “on the basis of image analysis” can include the meaning of “on the basis of a process using a pixel value constituting image data”.

According to another aspect, in the medical image processing device, the one or more processors may determine a contrast state of the image on the basis of the estimated elapsed period.

According to this aspect, it is possible to determine the contrast state on the basis of the estimated elapsed period from the start of the injection of the contrast agent.

The determination of the contrast state can include determination of a contrast time phase.

According to still another aspect, in the medical image processing device, the one or more processors may receive an input of at least one of a slice image, a partial image included in a three-dimensional image, a generated image generated on the basis of the partial image included in the three-dimensional image, or the three-dimensional image as the image.

According to this aspect, it is possible to estimate the elapsed period from the start of the injection of the contrast agent on the basis of the image analysis of at least one of the slice image, the partial image included in the three-dimensional image, the generated image generated on the basis of the partial image included in the three-dimensional image, or the three-dimensional image.

According to yet another aspect, in the medical image processing device, the one or more processors may estimate the elapsed period using a trained regression model.

According to this aspect, the trained regression model can be used to perform the estimation of the elapsed period in which a certain level of estimation accuracy is ensured.

According to still yet another aspect, in the medical image processing device, the one or more processors may receive an input of a first image, receive an input of a second image that belongs to the same image series as the first image and that is captured at a position different from that of the first image, estimate a first elapsed period which is an elapsed period from the start of the injection of the contrast agent in the first image, estimate a second elapsed period which is an elapsed period from the start of the injection of the contrast agent in the second image, and estimate the elapsed period belonging to the image series on the basis of the first elapsed period and the second elapsed period.

According to this aspect, it is possible to ensure certain robustness for the estimated elapsed period.

For example, even in a case where the first image or the second image is an image in which it is difficult to estimate the elapsed period, it is possible to estimate the elapsed period in which certain reliability is ensured.

According to yet still another aspect, in the medical image processing device, the one or more processors may integrate the first elapsed period and the second elapsed period to estimate the elapsed period.

According to still yet another aspect, in the medical image processing device, the one or more processors may estimate the first elapsed period and the second elapsed period using a trained regression model.

According to this aspect, the trained regression model can be used to estimate the first elapsed period and the second elapsed period in which a certain level of estimation accuracy is ensured.

According to yet still another aspect, in the medical image processing device, the one or more processors may estimate an estimated value output from the regression model and a certainty of the output estimated value for each of the first image and the second image and may integrate estimation results for each of the first image and the second image on the basis of the estimated value and the certainty estimated for each of the first image and the second image using the regression model.

According to this aspect, a plurality of sets of the estimated values and the certainties of the estimated values based on the first image and the second image are obtained, the estimation results are integrated on the basis of the plurality of sets of the estimated values and the certainties of the estimated values, and an estimated value is obtained as the integration result. Therefore, during the integration, each certainty is considered, and a certain level of accuracy is ensured for the integrated estimation result.

The estimation may include the concept of inference and prediction.

The certainty can include the concept of a certainty factor and a reliability degree.

According to still yet another aspect, in the medical image processing device, the one or more processors may estimate a probability distribution having the estimated value as a random variable for each of the first image and the second image on the basis of the estimated value and the certainty of the estimated value, integrate the probability distributions of the first image and the second image to generate an integrated distribution, and specify a final estimated value on the basis of the integrated distribution.

According to this aspect, it is possible to derive the estimated value of the elapsed period based on a plurality of random variables estimated from the first image and the second image.

According to yet still another aspect, in the medical image processing device, the one or more processors may estimate a probability distribution having the estimated value as a random variable for each of the first image and the second image on the basis of the estimated value and the certainty of the estimated value and specify a value at which a product of probabilities at the same random variable is maximized on the basis of the probability distribution of each of the first image and the second image.

According to this aspect, a value at which a simultaneous probability is maximized is specified on the basis of a plurality of probability distributions. Therefore, it is possible to derive the estimated value of the elapsed period with high accuracy in consideration of the certainty estimated according to the input image.

According to still yet another aspect, in the medical image processing device, the one or more processors may perform variable conversion to convert the estimated value output from the regression model into a first parameter of a probability distribution model and perform variable conversion to convert a value indicating the certainty output from the regression model into a second parameter of the probability distribution model.

According to yet still another aspect, in the medical image processing device, the probability distribution model may be a Laplace distribution.

According to still yet another aspect, in the medical image processing device, the probability distribution model may be a Gaussian distribution.

According to still yet another aspect, in the medical image processing device, the one or more processors may perform logarithmic conversion to take a logarithm of the probability distribution, calculate a sum of logarithmic probability densities corresponding to the probability distributions of the first image and the second image during the integration, and calculate a value at which a simultaneous logarithmic probability density is maximized.

According to yet still another aspect, in the medical image processing device, the regression model may include a trained model generated by performing machine learning using training data in which an image for input and a teaching signal are associated with each other.

According to still yet another aspect, in the medical image processing device, the regression model may be configured using a convolutional neural network.

According to yet still another aspect, in the medical image processing device, the first image and the second image may be slice images included in the same image series.

According to still yet another aspect, in the medical image processing device, the first image and the second image may include different partial images included in a three-dimensional image.

According to yet still another aspect, in the medical image processing device, the first image and the second image may include generated images that are generated on the basis of different partial images included in a three-dimensional image.

According to still yet another aspect, in the medical image processing device, the first image and the second image may include three-dimensional images.

The three-dimensional image or the like is used as the input image. Therefore, it is possible to suppress the deterioration of the estimation accuracy of the elapsed period and to speed up the process of estimating the elapsed period.

According to the present disclosure, there is provided a medical image processing method comprising a computer to receive an input of an image generated by performing contrast imaging and to estimate an elapsed period from start of injection of a contrast agent in the image on the basis of image analysis of the image.

According to the medical image processing method of the present disclosure, it is possible to obtain the same operation and effect as those of the medical image processing device according to the present disclosure. Configuration requirements of a medical image processing device according to another aspect can be applied to configuration requirements of a medical image processing method according to another aspect.

According to the present disclosure, there is provided a program causing a computer to implement: a function of receiving an input of an image generated by performing contrast imaging; and a function of estimating an elapsed period from start of injection of a contrast agent in the image on the basis of image analysis of the image.

According to the program of the present disclosure, it is possible to obtain the same operation and effect as those of the medical image processing device according to the present disclosure. Components of a medical image processing device according to another aspect can be applied to components of a program according to another aspect.

According to the present invention, it is possible to estimate the elapsed period from the start of the injection of the contrast agent in the image on the basis of the image analysis of the input image. Therefore, even in a case where it is difficult to use accessory information of the image such as the imaging start time point, it is possible to estimate the elapsed period from the start of the injection of the contrast agent in the image.

### BRIEF DESCRIPTION OF THE DRAWINGS

FIG. 1 is a conceptual diagram illustrating an outline of a process used in a contrast state determination device according to a first embodiment.

FIG. 2 is a block diagram schematically illustrating an example of a hardware configuration of the contrast state determination device according to the first embodiment.

FIG. 3 is a functional block diagram illustrating an outline of processing functions of the contrast state determination device according to the first embodiment.

FIG. 4 is a flowchart illustrating a procedure of a contrast state determination method according to the first embodiment.

FIG. 5 is a conceptual diagram illustrating an outline of a process used in a contrast state determination device according to a second embodiment.

FIG. 6 is a functional block diagram illustrating an outline of processing functions of a number-of-seconds estimation device used in the contrast state determination device according to the second embodiment.

FIG. 7 is a flowchart illustrating a procedure of a contrast state determination method according to the second embodiment.

FIG. 8 is a conceptual diagram illustrating an outline of a process used in a contrast state determination device according to a third embodiment.

FIG. 9 is a diagram illustrating Example 1 of a process of a number-of-seconds distribution estimation unit.

FIG. 10 is a graph illustrating an example of a function used for variable conversion.

FIG. 11 illustrates an example of a graph of a number-of-seconds distribution that is estimated on the basis of a parameter u and a parameter b estimated by a number-of-seconds distribution estimation unit.

FIG. 12 is a diagram illustrating an example of processes of an integration unit and a maximum point specification unit.

FIG. 13 is a diagram schematically illustrating an example of a machine learning method for generating a regression model that is used in the number-of-seconds distribution estimation unit.

FIG. 14 is a diagram illustrating a loss function used during training.

FIG. 15 is a block diagram schematically illustrating an example of a hardware configuration of a contrast state determination device according to a third embodiment.

FIG. 16 is a functional block diagram illustrating an outline of processing functions of a regression estimation device used in the contrast state determination device according to the third embodiment.

FIG. 17 is a diagram illustrating Example 2 of a process of a number-of-seconds distribution estimation unit of a regression estimation device provided in a contrast state determination device according to a fourth embodiment.

FIG. 18 is a graph of a number-of-seconds distribution that is estimated on the basis of a parameter μ and a parameter σ² estimated by using the number-of-seconds distribution estimation unit.

FIG. 19 is a diagram illustrating an example of processes of an integration unit and a maximum point specification unit of the regression estimation device provided in the contrast state determination device according to the fourth embodiment.

FIG. 20 is a diagram schematically illustrating an example of a machine learning method for generating a regression model that is used in the number-of-seconds distribution estimation unit provided in the contrast state determination device according to the fourth embodiment.

FIG. 21 is a diagram illustrating Modification Example 1 of the image used for input to the number-of-seconds estimation device.

FIG. 22 is a diagram illustrating Modification Example 2 of the image used for input to the number-of-seconds estimation device.

FIG. 23 is a block diagram illustrating an example of a configuration of a medical information system in which the contrast state determination device is used.

### DESCRIPTION OF THE PREFERRED EMBODIMENTS

Hereinafter, preferred embodiments of the present invention will be described in detail with reference to the accompanying drawings. In the specification, the same components are denoted by the same reference numerals, and the duplicate description thereof will be omitted as appropriate.

### Contrast State Determination Device According to First Embodiment

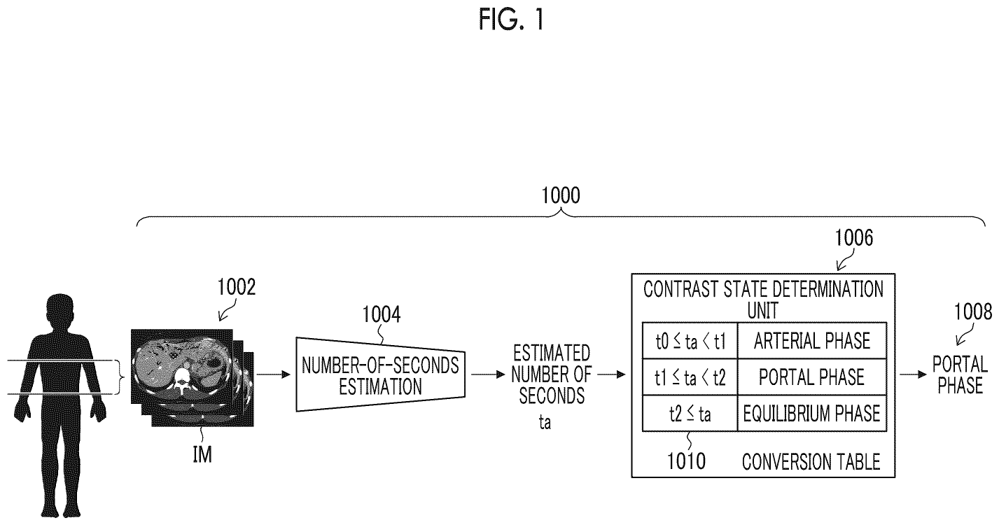

FIG. 1 is a conceptual diagram illustrating an outline of a process used in a contrast state determination device according to a first embodiment. A contrast state determination device 1000 illustrated in FIG. 1 acquires any one slice image IM included in a plurality of slice images sampled at equal intervals from three-dimensional CT data of a patient captured by a CT apparatus and determines a contrast state such as a contrast time phase of the slice image IM.

That is, the contrast state determination device 1000 estimates the number of seconds ta of the acquired slice image IM from the injection of a contrast agent and determines the contrast state of the acquired slice image IM on the basis of the estimated number of seconds ta.

In addition, the slice image described in the embodiment is an example of an image generated by performing contrast imaging. The number of seconds ta from the injection of the contrast agent described in the embodiment is an example of an elapsed period from the start of the injection of the contrast agent.

Hereinafter, unless otherwise specified, the number of seconds includes the meaning of the number of seconds indicating the elapsed time from the injection of the contrast agent. The elapsed time is synonymous with an elapsed period. In addition, the slice image may be paraphrased as a tomographic image. That is, the slice image may be understood as a cross-sectional image which is substantially a two-dimensional image.

Examples of the contrast time phase of the liver include an arterial phase, a portal phase, and an equilibrium phase. Examples of the contrast time phase of the kidney include a corticomedullary phase, a parenchymal phase, and an excretory phase. The contrast state can include a non-contrast phase.

The contrast state determination device 1000 can be implemented using hardware and software of a computer. The contrast state determination device 1000 comprises an image receiving unit 1002, a number-of-seconds estimation unit 1004, a contrast state determination unit 1006, and an output unit 1008.

The image receiving unit 1002 receives the slice image IM. FIG. 1 illustrates an example of the image receiving unit 1002 that receives any one slice image IM included in a plurality of slice images. The reception of the slice image IM is synonymous with acquisition of the slice image IM. The image receiving unit 1002 may receive a plurality of slice images IM. In addition, an aspect in which a plurality of slice images IM are received will be described in detail below.

The number-of-seconds estimation unit 1004 estimates the number of seconds ta on the basis of image analysis of the slice image IM and outputs the estimated number of seconds ta as an estimated number-of-seconds value. For example, the number of seconds ta can be estimated on the basis of pixel values constituting the slice image IM. In addition, the term “slice image” may include the meaning of image data which is a signal indicating the slice image.

The number-of-seconds estimation unit 1004 can use a trained model that has been trained by machine learning. An example of a learning model is a convolutional neural network. The convolutional neural network is referred to as a CNN using an abbreviation of Convolutional Neural Network.

The number-of-seconds estimation unit 1004 can use a trained model that has been trained using, as learning data, a set of a slice image, of which the number of seconds is known in advance, and the number of seconds corresponding to the slice image.

The contrast state determination unit 1006 acquires the number of seconds ta output from the number-of-seconds estimation unit 1004 and separates the acquired number of seconds ta to obtain a final contrast state. That is, the contrast state determination unit 1006 determines the contrast state of the slice image IM to be processed, on the basis of the acquired estimated number-of-seconds value. FIG. 1 illustrates the contrast state determination unit 1006 that determines the contrast state using a conversion table 1010. The conversion table 1010 illustrated in FIG. 1 is a table that defines a correspondence relationship between the number of seconds ta output from the number-of-seconds estimation unit 1004 and the classification of the contrast state.

In the conversion table 1010, t₀ can be set to 0 seconds, t₁ can be set to 50 seconds, and t₂ can be set to 120 seconds. That is, the contrast state determination unit 1006 can determine the contrast state to be the arterial phase in a case where the number of seconds ta is less than 50 seconds, determine the contrast state to be the portal phase in a case where the number of seconds ta is equal to or greater than 50 seconds and less than 120 seconds, and determine the contrast state to be the equilibrium phase in a case where the number of seconds ta is equal to or greater than 120 seconds.

The contrast state determination device 1000 can comprise a storage device that stores the conversion table 1010. The contrast state determination device 1000 may acquire the conversion table 1010 from an external database storage device.

The output unit 1008 outputs information of the contrast state determined by the contrast state determination unit 1006. For example, in a case where the number of seconds ta output from the number-of-seconds estimation unit 1004 is 72 seconds and the contrast state of the acquired slice image IM is determined to be the portal phase, the output unit 1008 outputs the portal phase as a determination result.

### [Description of Medical Image Used for Input]

In a DICOM standard that defines a format of a medical image and a communication protocol, a series ID is defined in a unit called a study ID which is an identification code for specifying an examination type. In addition, ID is an abbreviation of identification. Further, the medical image is synonymous with a medicine image.

For example, in a case where liver contrast imaging is performed on a certain patient, CT imaging is performed in a range including the liver a plurality of times while changing an imaging timing. As an example of a plurality of imaging operations, a first imaging operation is performed before the injection of the contrast agent, a second imaging operation is performed 35 seconds after the injection of the contrast agent, a third imaging operation is performed 70 seconds after the injection of the contrast agent, and a fourth imaging operation is performed 180 seconds after the injection of the contrast agent.

The four imaging operations are performed to obtain four types of CT data. The CT data referred to here is three-dimensional data composed of a plurality of consecutive slice images and is an aggregate of the plurality of slice images constituting the three-dimensional data, and the aggregate of the plurality of slice images is referred to as an image series.

The same study ID and different series IDs are given to the four types of CT data obtained by performing a series of imaging operations including the four imaging operations.

For example, study 1 is given as a study ID for an examination of liver contrast imaging on a specific patient, and a unique ID is given to each series as follows: series 1 is given as a series ID to CT data obtained by imaging before the injection of the contrast agent; series 2 is given to CT data obtained by imaging 35 seconds after the injection of the contrast agent; series 3 is given to CT data obtained by imaging 70 seconds after the injection of the contrast agent; and series 4 is given to CT data obtained by imaging 180 seconds after the injection of the contrast agent.

Therefore, the CT data can be identified by combining the study ID and the series ID. Meanwhile, in some cases, in the actual CT data, the correspondence relationship between the series ID and the imaging timing is not clearly understood. The imaging timing referred to here may be read as the elapsed time from the injection of the contrast agent.

In addition, the size of the three-dimensional CT data is large. Therefore, in a case where a process, such as the estimation of the number of seconds, is performed using the CT data as input data without any change, it may be difficult to process the CT data from the viewpoint of a processing period, a processing load, and the like. Therefore, the contrast state determination device 1000 estimates the number of seconds on the basis of image analysis, using one or more slice images in the same image series as an input.

### [Example of Hardware Configuration]

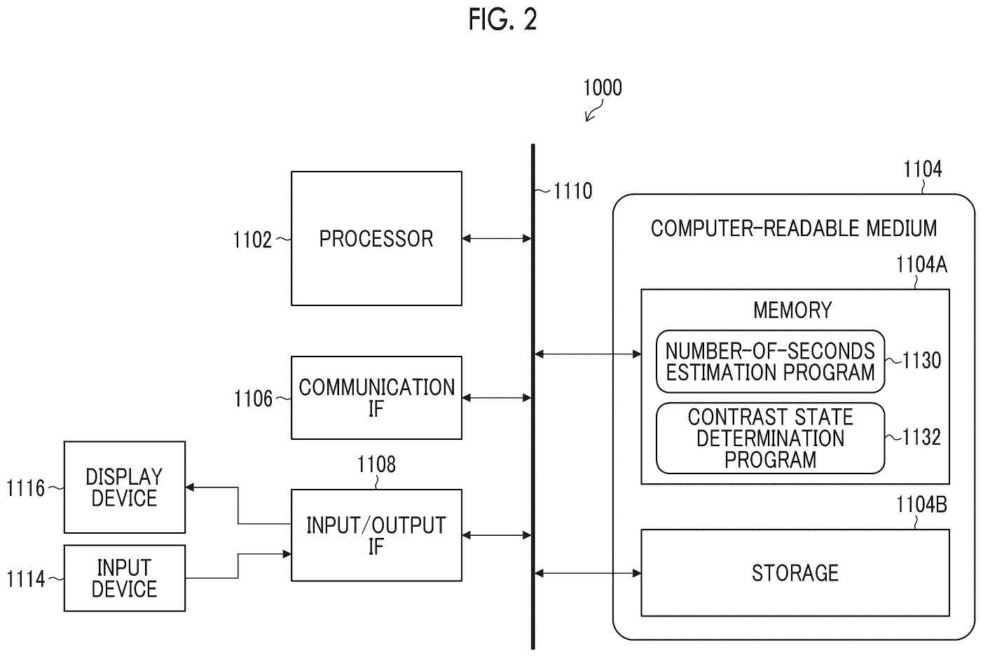

FIG. 2 is a block diagram schematically illustrating an example of a hardware configuration of the contrast state determination device according to the first embodiment. The contrast state determination device 1000 can be implemented by a computer system that is configured using one or a plurality of computers. Here, an example will be described in which one computer executes a program to implement various functions of the contrast state determination device 1000.

In addition, the form of the computer that functions as the contrast state determination device 1000 is not particularly limited, and the computer may be, for example, a server computer, a workstation, a personal computer, or a tablet terminal.

The contrast state determination device 1000 comprises a processor 1102, a computer-readable medium 1104 which is a non-transitory tangible object, a communication interface 1106, an input/output interface 1108, and a bus 1110.

The processor 1102 includes a central processing unit (CPU). The processor 1102 may include a graphics processing unit (GPU). The processor 1102 is connected to the computer-readable medium 1104, the communication interface 1106, and the input/output interface 1108 through the bus 1110. The processor 1102 reads, for example, various programs and data stored in the computer-readable medium 1104 and performs various processes.

The computer-readable medium 1104 includes a memory 1104A which is a main storage device and a storage 1104B which is an auxiliary storage device. The storage 1104B can be configured using a hard disk apparatus, a solid state drive device, an optical disk, a magneto-optical disk, and a semiconductor memory. The storage 1104B can be configured using an appropriate combination of a hard disk apparatus and the like. For example, various programs and data are stored in the storage 1104B.

In addition, the hard disk apparatus can be referred to as an HDD which is an abbreviation of Hard Disk Drive in English. Further, the solid state drive apparatus can be referred to as an SSD which is an abbreviation of Solid State Drive in English.

The memory 1104A is used as a work area of the processor 1102 and is used as a storage unit that temporarily stores the program and various types of data read out from the storage 1104B. The program stored in the storage 1104B is loaded into the memory 1104A, and commands of the program are executed using the processor 1102 such that the processor 1102 functions as processing units that perform various processes defined by the program. The memory 1104A stores, for example, a number-of-seconds estimation program 1130, a contrast state determination program 1132, and various types of data executed by the processor 1102.

The number-of-seconds estimation program 1130 includes a trained model that has been trained using machine learning and causes the processor 1102 to perform a number-of-seconds estimation process described with reference to FIG. 1. Similarly, the contrast state determination program 1132 includes a trained model which has been trained using machine learning and causes the processor 1102 to perform a contrast state determination process described with reference to FIG. 1. In addition, the number-of-seconds estimation program 1130 and the contrast state determination program 1132 may be configured as one program.

The communication interface 1106 performs a communication process with an external device wirelessly or in a wired manner to exchange information with the external device. The contrast state determination device 1000 is connected to a communication line through the communication interface 1106. The communication line may be a local area network or a wide area network. The communication interface 1106 can play a role of a data acquisition unit that receives the input of data such as an image. In addition, the communication line is not illustrated.

The contrast state determination device 1000 can comprise an input device 1114 and a display device 1116. The input device 1114 and the display device 1116 are connected to the bus 1110 through the input/output interface 1108. Examples of the input device 1114 include a keyboard, a mouse, a multi-touch panel, other pointing devices, and a voice input device. The input device 1114 may be an appropriate combination of the keyboard and the like.

The display device 1116 is an output interface on which various types of information are displayed. Examples of the display device 1116 include a liquid crystal display, an organic EL display, and a projector. The display device 1116 may be an appropriate combination of the liquid crystal display and the like. In addition, the organic EL is referred to as an OEL which is an abbreviation of Organic Electro-Luminescence in English.

### [Functional Configuration of Contrast State Determination Device]

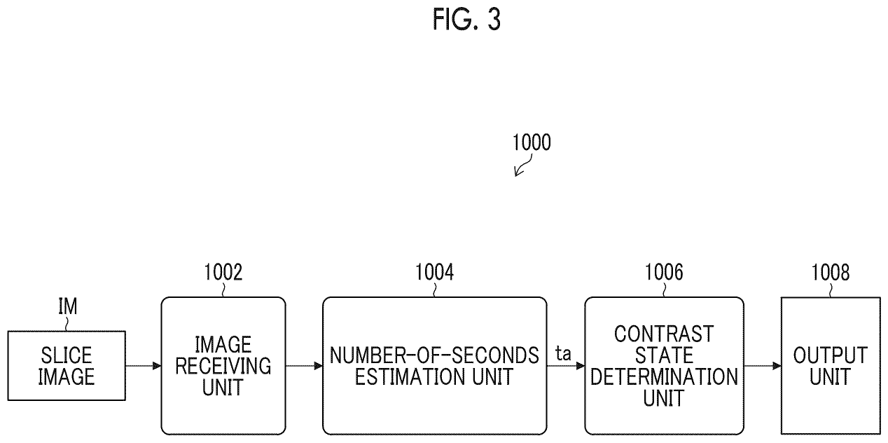

FIG. 3 is a functional block diagram illustrating an outline of processing functions of the contrast state determination device according to the first embodiment. The processor 1102 illustrated in FIG. 2 executes the number-of-seconds estimation program 1130 stored in the memory 1104A to function as the image receiving unit 1002 and the number-of-seconds estimation unit 1004 and executes the contrast state determination program 1132 to function as the contrast state determination unit 1006 and the output unit 1008.

The image receiving unit 1002 acquires a slice image sampled from CT data as the slice image IM to be processed. The image receiving unit 1002 may perform a process of cutting out the slice images IM from the CT data at equal intervals or may acquire the slice images IM sampled in advance using a processing unit (not shown) or the like.

The slice image IM acquired through the image receiving unit 1002 is input to the number-of-seconds estimation unit 1004. The number-of-seconds estimation unit 1004 outputs an estimated number-of-seconds value based on the image analysis of the input slice image IM. The estimated number-of-seconds value output from the number-of-seconds estimation unit 1004 is input to the contrast state determination unit 1006.

The contrast state determination unit 1006 acquires the estimated number-of-seconds value output from the number-of-seconds estimation unit 1004, determines the contrast state of the slice image IM to be processed on the basis of the acquired estimated number-of-seconds value, outputs a determination result through the output unit 1008.

The output unit 1008 is an output interface that displays the contrast state of the slice image IM to be processed. The output unit 1008 may function as an output interface that provides the contrast state of the slice image IM to be processed to other processing units. The output unit 1008 may include at least one processing unit that performs, for example, a process of generating data for display and a data conversion process for transmission of data to the outside or the like. The contrast state determined by the contrast state determination device 1000 may be displayed by the display device or the like.

The contrast state determination device 1000 may be incorporated into a medical image processing device for processing a medical image acquired in a medical institution such as a hospital. In addition, the processing functions of the contrast state determination device 1000 may be provided as a cloud service.

### [Procedure of Contrast State Determination Method]

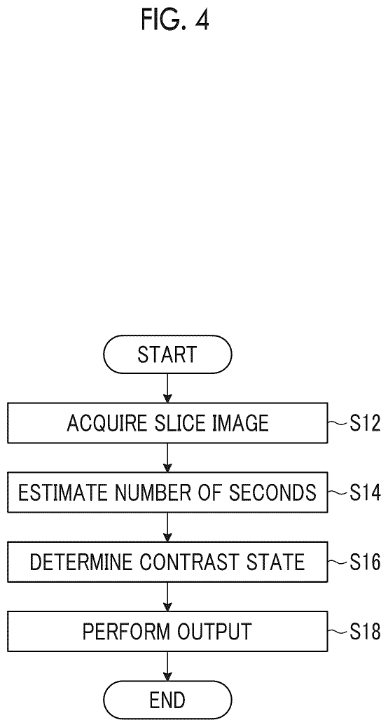

FIG. 4 is a flowchart illustrating a procedure of a contrast state determination method according to the first embodiment. In an image acquisition step S12, the image receiving unit 1002 illustrated in FIG. 3 acquires the slice image IM. After the image acquisition step S12, the process proceeds to a number-of-seconds estimation step S14.

In the number-of-seconds estimation step S14, the number-of-seconds estimation unit 1004 estimates the number of seconds ta of the acquired slice image IM on the basis of the image analysis of the slice image IM and outputs the estimated number of seconds ta as the estimated number-of-seconds value. After the number-of-seconds estimation step S14, the process proceeds to a contrast state determination step S16.

In the contrast state determination step S16, the contrast state determination unit 1006 determines a contrast state corresponding to the estimated number-of-seconds value of the slice image IM using the conversion table 1010 illustrated in FIG. 1. After the contrast state determination step S16, the process proceeds to an output step S18.

In the output step S18, the output unit 1008 outputs the contrast state of the slice image IM determined in the contrast state determination step S16. After the output step S18, the procedure of the contrast state determination method is ended.

Waiting for the input of the next slice image IM may be performed after the output step S18. In a case where the next slice image IM is input, each step from the image acquisition step S12 to the output step S18 may be performed. Waiting for the input of the next slice image IM may be performed after the output step S18. In a case where the next slice image IM is not input in a predetermined period, the procedure of the contrast state determination method may be ended.

### [Example of Application to Number-of-Seconds Estimation Device and Number-of-Seconds Estimation Method]

The contrast state determination device according to the first embodiment can function as a number-of-seconds estimation device that acquires the slice image IM and estimates the number of seconds ta of the slice image IM on the basis of the image analysis of the slice image IM. The number-of-seconds estimation device comprises the image receiving unit 1002 and the number-of-seconds estimation unit 1004 illustrated in FIG. 3, and the processor 1102 illustrated in FIG. 2 can execute the number-of-seconds estimation program 1130 to implement an image receiving function and a number-of-seconds estimation function.

Further, the number-of-seconds estimation device can cause a computer to perform a number-of-seconds estimation method including the image acquisition step S12, the number-of-seconds estimation step S14, and the output step S18 illustrated in FIG. 4.

### Operation and Effect of First Embodiment

The contrast state determination device 1000 according to the first embodiment can obtain the following operation and effect.

[1]

The number of seconds ta of the slice image IM from the injection of the contrast agent is estimated on the basis of the image analysis of the acquired slice image IM. Therefore, for example, in a case where information related to the imaging time point or the like is not included in a DICOM tag or in a case where the information related to the imaging time point or the like included in the DICOM tag is incorrect, it is possible to estimate the number of seconds ta from the injection of the contrast agent.

[2]

The contrast state of the acquired slice image IM is determined on the basis of the estimated number-of-seconds value. This makes it possible to determine the contrast state on the basis of the estimated number-of-seconds value which is the estimated value of the number of seconds ta from the injection of the contrast agent.

[3]

The contrast state is determined using the conversion table 1010 that defines the correspondence relationship between the number of seconds ta output from the number-of-seconds estimation unit 1004 and the classification of the contrast state. This makes it possible to perform the conversion of the estimated number-of-seconds value into the contrast state based on the conversion table 1010.

In addition, the contrast state determination device 1000 according to the first embodiment is an example of a medical image processing device. The same applies to contrast state determination devices according to second to fourth embodiments which will be described below. Further, the contrast state determination method according to the first embodiment is an example of a medical image processing method. The same applies to contrast state determination methods used in the second to fourth embodiments which will be described below.

### Contrast State Determination Device According to Second Embodiment

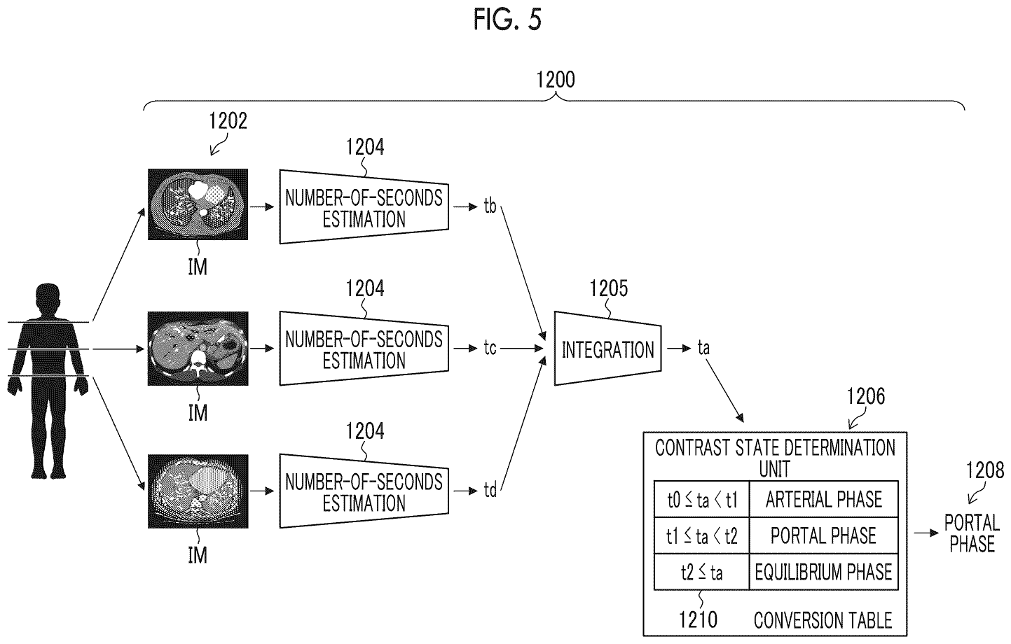

FIG. 5 is a conceptual diagram illustrating an outline of a process used in the contrast state determination device according to the second embodiment. Hereinafter, components different from those in the first embodiment will be mainly described, and description of components common to the first embodiment will be omitted as appropriate.

A contrast state determination device 1200 according to the second embodiment acquires a plurality of slice images IM sampled from CT images at equal intervals and estimates the number of seconds for each of the plurality of slice images IM.

The contrast state determination device 1200 performs an integration process, such as an averaging process, on a plurality of estimated number-of-seconds values to determine an integrated number of seconds ta. The contrast state determination device 1200 separates the determined number of seconds ta to obtain the final contrast state of the plurality of slice images IM.

The contrast state determination device 1200 comprises an image receiving unit 1202, a number-of-seconds estimation unit 1204, an integration unit 1205, a contrast state determination unit 1206, and an output unit 1208. The image receiving unit 1202 acquires the plurality of slice images IM. FIG. 5 illustrates an aspect in which three slice images IM are acquired as the plurality of slice images IM.

The number-of-seconds estimation unit 1204 illustrated in FIG. 5 estimates the number of seconds tb, the number of seconds tc, and the number of seconds td for the three slice images IM, respectively. The integration unit 1205 performs a process of integrating the number of seconds tb, the number of seconds tc, and the number of seconds td to determine the number of seconds ta.

The integration unit 1205 may perform a weighted averaging process as the integration process. For example, in a case where the number of seconds tb is 70 seconds, the number of seconds tc is 75 seconds, and the number of seconds td is 80 seconds, the integration unit 1205 can determine the number of seconds ta to be 72 seconds. The integration unit 1205 may perform, for example, an arithmetic averaging process as the integration process.

The contrast state determination unit 1206 determines the contrast state of the plurality of slice images IM on the basis of the number of seconds ta determined by the integration process. The contrast state determination unit 1206 illustrated in FIG. 5 determines the contrast states of the plurality of slice images IM with reference to a conversion table 1210.

The output unit 1208 outputs the contrast state of the plurality of slice images IM determined by the contrast state determination unit 1206. The output unit 1208 illustrated in FIG. 5 outputs, as the contrast state of the plurality of slice images IM, that the time phase of the plurality of slice images IM is the portal phase. In addition, any one of the plurality of slice images MI described in the embodiment is an example of a first image, and any one slice image different from the first image is an example of a second image whose imaging position is different from that of the first image.

### [Example of Hardware Configuration]

The contrast state determination device 1200 according to the second embodiment can have the hardware configuration illustrated in FIG. 2. Here, the description of the hardware configuration of the contrast state determination device 1200 will be omitted.

### [Functional Configuration of Contrast State Determination Device]

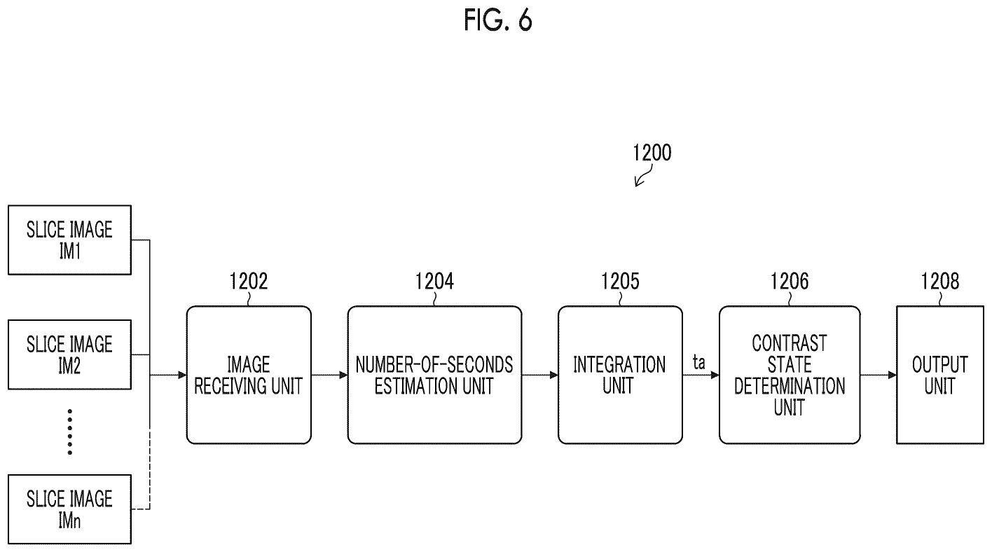

FIG. 6 is a functional block diagram illustrating an outline of a processing function of a number-of-seconds estimation device used in the contrast state determination device according to the second embodiment. The image receiving unit 1202 illustrated in FIG. 6 acquires a plurality of slice images IMi sampled from CT data. A subscript i indicates an index number for identifying the plurality of slice images.

FIG. 6 illustrates that n different slice images IMi (where i is 1 to n) can be input. n may be an integer equal to or greater than 2. That is, the image receiving unit 1202 can acquire two or more slice images IMi included in the same image series.

The number-of-seconds estimation unit 1204 estimates the number of seconds for each of the n slice images IMi included in the same image series on the basis of image analysis. The integration unit 1205 integrates n estimated number-of-seconds values corresponding to each of the n slice images IMi and outputs the number of seconds ta which is the estimated number-of-seconds value of the image series.

The contrast state determination unit 1206 determines the contrast state of the image series on the basis of the estimated number-of-seconds value determined as the result of the integration process. The output unit 1208 outputs the contrast state of the image series.

### [Procedure of Contrast State Determination Method]

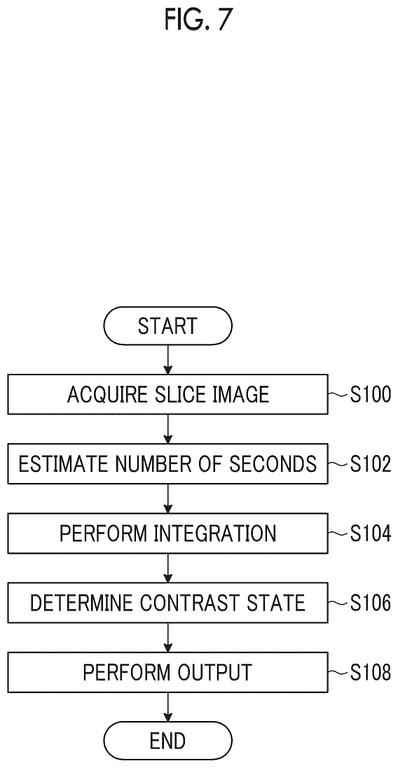

FIG. 7 is a flowchart illustrating a procedure of a contrast state determination method according to the second embodiment. In an image acquisition step S100, the image receiving unit 1202 illustrated in FIG. 6 acquires n slice images IMi. After the image acquisition step S100, the process proceeds to a number-of-seconds estimation step S102.

In the number-of-seconds estimation step S102, the number-of-seconds estimation unit 1204 estimates the number of seconds for each of the n slice images IMi on the basis of image analysis. After the number-of-seconds estimation step S102, the process proceeds to an integration step S104.

In the integration step S104, the integration unit 1205 integrates n estimated number-of-seconds values corresponding to each of the n slice images IMi to determine the estimated number-of-seconds value of the image series. After the integration step S104, the process proceeds to a contrast state determination step S106.

In the contrast state determination step S106, the contrast state determination unit 1206 determines the contrast state of the image series on the basis of the estimated number-of-seconds value determined as the result of the integration process. After the contrast state determination step S106, the process proceeds to an output step S108.

In the output step S108, the output unit 1208 outputs the contrast state of the image series. In the output step S108, after the contrast state of the image series including the n slice images IMi is output, the procedure of the contrast state determination method is ended.

In addition, the plurality of slice images IMi described in the embodiment are an example of the first image and the second image. Further, the number of seconds estimated for each of the n slice images IMi described in the embodiment on the basis of the image analysis is an example of a first elapsed period and a second elapsed period.

### Operation and Effect of Second Embodiment

The contrast state determination device 1200 according to the second embodiment can obtain the following operation and effect.

[1]

The number of seconds from the injection of the contrast agent is estimated for each of the plurality of slice images IMi included in the same image series on the basis of the image analysis of the plurality of slice images IMi. A plurality of numbers of seconds are integrated, and the number of seconds ta of the image series is determined.

This makes it possible to weight and integrate the estimation results corresponding to each of the plurality of acquired slice images IMi, to reduce the influence of an image in which it is difficult to estimate the number of seconds, such as an image that includes an artifact and makes it difficult to perform scene analysis, and to obtain an estimated value with high accuracy.

For example, in a case where a slice image that is inappropriate for estimation is received as one of the inputs, even though the estimated number-of-seconds value corresponding to the input deviates significantly, a certainty is reduced, and the influence on the integration result is suppressed.

[2]

The contrast state of the image series is determined on the basis of the integrated estimated number-of-seconds value. This makes it possible to determine the contrast state of the image series on the basis of the plurality of acquired slice images IMi.

### Contrast State Determination Device according to Third Embodiment

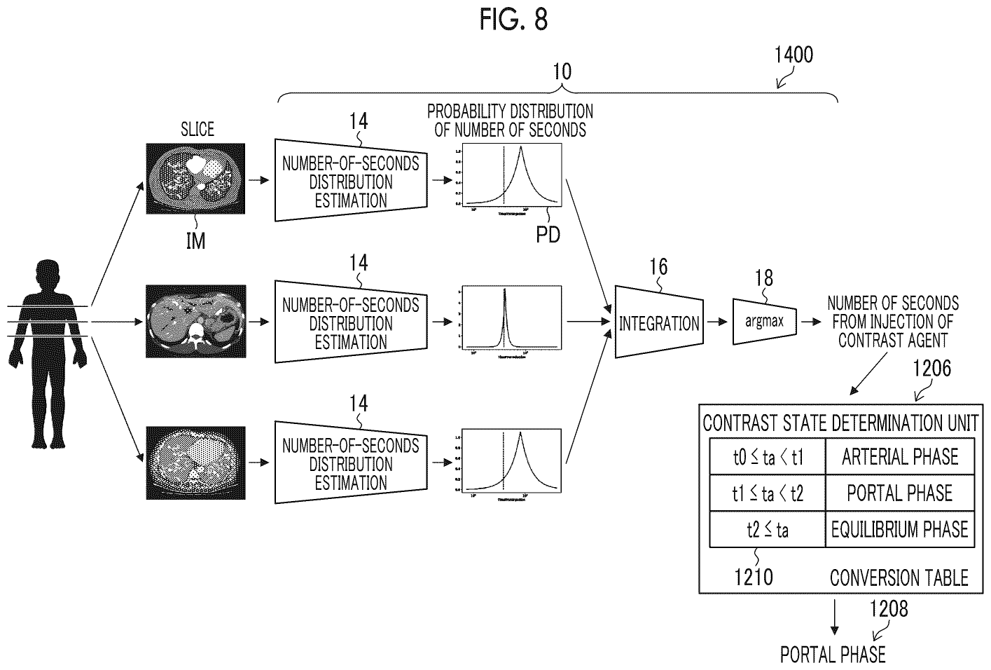

FIG. 8 is a conceptual diagram illustrating an outline of a process used in a contrast state determination device according to a third embodiment. A contrast state determination device 1400 according to the third embodiment comprises a regression estimation device 10 instead of the image receiving unit 1202, the number-of-seconds estimation unit 1204, and the integration unit 1205 of the contrast state determination device 1200 illustrated in FIG. 5.

The regression estimation device 10 comprises a data acquisition unit 12 that receives an input of a plurality of slice images IM, a number-of-seconds distribution estimation unit 14 that estimates a number-of-seconds distribution, which is a probability distribution of the number of seconds, from the plurality of acquired slice images IM, and an integration unit 16 that integrates a plurality of number-of-seconds distributions PD estimated from the plurality of slice images IM.

In addition, the regression estimation device 10 comprises a maximum point specification unit 18 that specifies the number of seconds at which a probability is maximized from an integrated distribution which is a new distribution obtained by the integration process. The number of seconds at which the probability is maximized specified by the maximum point specification unit 18 is output as a final result.

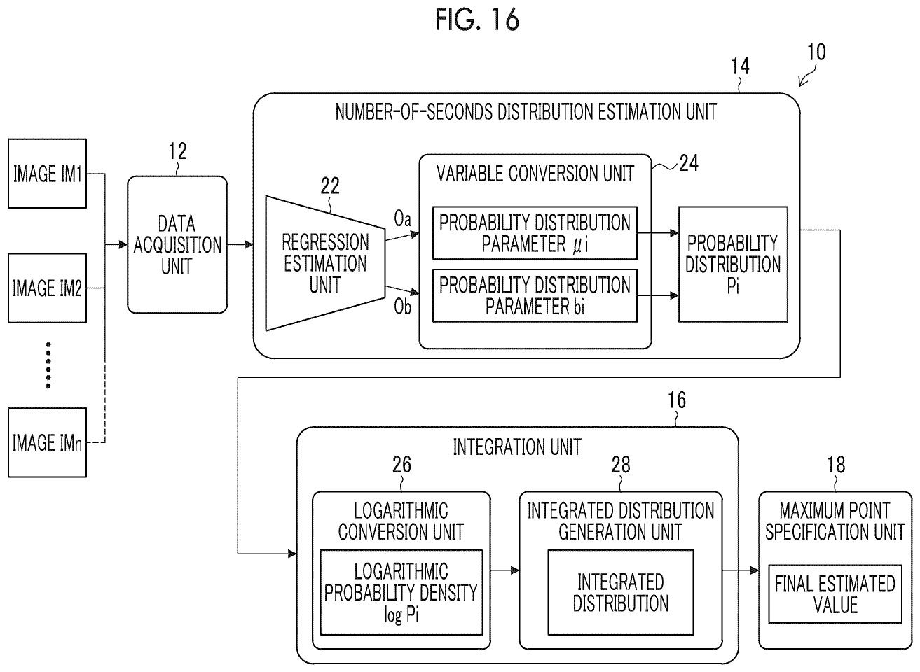

In addition, the data acquisition unit 12 is not illustrated in FIG. 8. The data acquisition unit 12 is illustrated in FIG. 16. Further, in order to illustrate a flow of a process in a case where three different slice images IM are input, three number-of-seconds distribution estimation units 14 are illustrated in FIG. 8. However, the number-of-seconds distribution estimation unit 14 to which each slice image IM is input is the same and is a single processing unit. Of course, the regression estimation device 10 may comprise a plurality of number-of-seconds distribution estimation units 14.

FIG. 8 illustrates an aspect in which the regression estimation device 10 acquires a plurality of slice images IM. However, the regression estimation device 10 may acquire one slice image IM and estimate the number of seconds from one slice image IM.

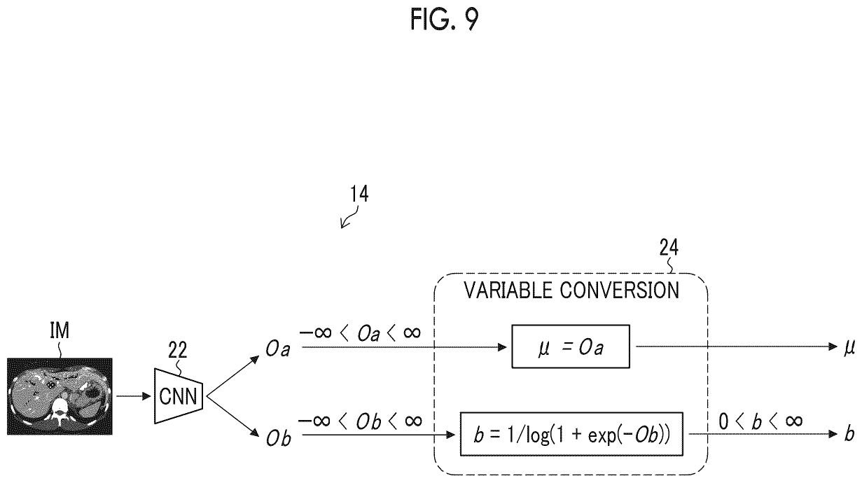

FIG. 9 is a diagram illustrating Example 1 of a process in the number-of-seconds distribution estimation unit. The number-of-seconds distribution estimation unit 14 comprises a regression estimation unit 22 and a variable conversion unit 24. The regression estimation unit 22 includes a trained model that has been trained by machine learning. The trained model receives the input of the slice image IM and outputs an estimated number-of-seconds value Oa and a score value Ob indicating the certainty of the estimated number-of-seconds value Oa. The trained model as a regression model used in the regression estimation unit 22 is configured by, for example, a convolutional neural network.

The numerical range of the estimated number-of-seconds value Oa output from the regression estimation unit 22 may be −∞<Oa<∞, and the numerical range of the score value Ob of the certainty may be −∞<Ob<∞. In addition, the score value Ob of the certainty may be read as a score value Ob of a certainty factor. The regression model is not limited to the CNN, and various machine learning models can be applied.

The variable conversion unit 24 performs variable conversion on the estimated number-of-seconds value Oa and the score value Ob of the certainty according to the following Expressions 1 and 2 to generate a parameter u and a parameter b of the probability distribution model, respectively.

μ=Oa Expression 1

b=1/log(1+exp(−Ob)) Expression 2

The function represented by Expression 2 is an example of mapping that converts the score value Ob of the certainty into a value b in a positive region.

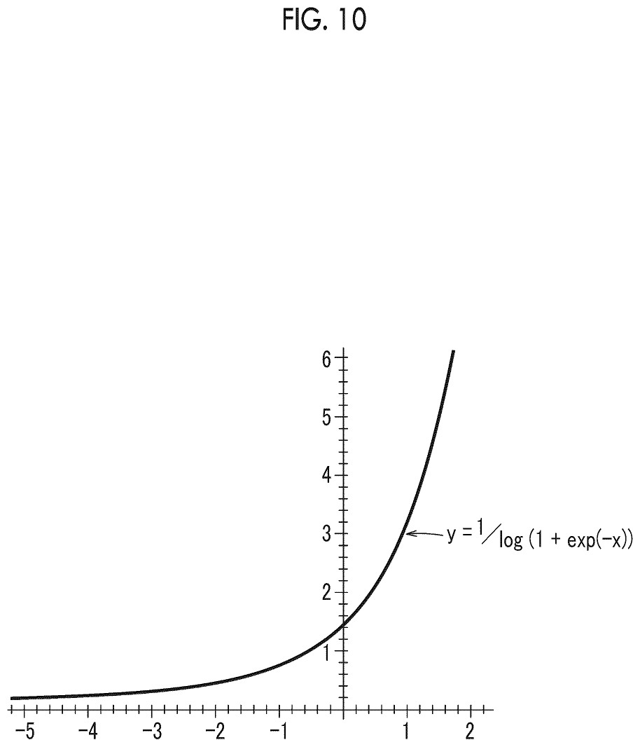

FIG. 10 is a graph illustrating an example of a function that is used for variable conversion. FIG. 10 illustrates a graph of a function y=1/log(1+exp(x)).

In the contrast state determination device 1400 according to the third embodiment, the Laplace distribution is applied as the probability distribution model of the number-of-seconds distribution. The Laplace distribution is represented as the function represented by Expression 3.

$$f ( x ; μ , b ) = \frac{1}{2 b} exp ( - \frac{\vert x - μ \vert}{b} ) \qquad Expression 3$$

The reason for converting the score value Ob of the certainty into the positive value b is related to the application of the Laplace distribution as the probability distribution model of the number-of-seconds distribution. In a case where the parameter b is a negative value that satisfies b<0, it is necessary to ensure that the Laplace distribution is not established as the probability distribution and the parameter b is a positive value that satisfies b>0.

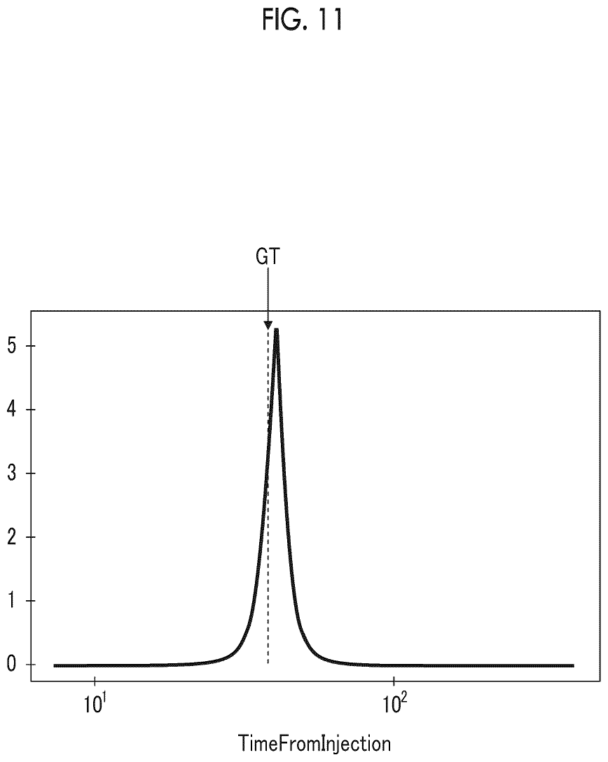

FIG. 11 illustrates an example of a graph of the number-of-seconds distribution that is estimated on the basis of the parameter u and the parameter b estimated by the number-of-seconds distribution estimation unit. In addition, a position indicated by a broken line GT in FIG. 11 corresponds to a correct answer number of seconds which is a correct number of seconds. The estimation of a set of the estimated number-of-seconds value Oa and the score value Ob of the certainty of the estimated number-of-seconds value Oa from the input slice image IM substantially corresponds to the estimation of the number-of-seconds distribution.

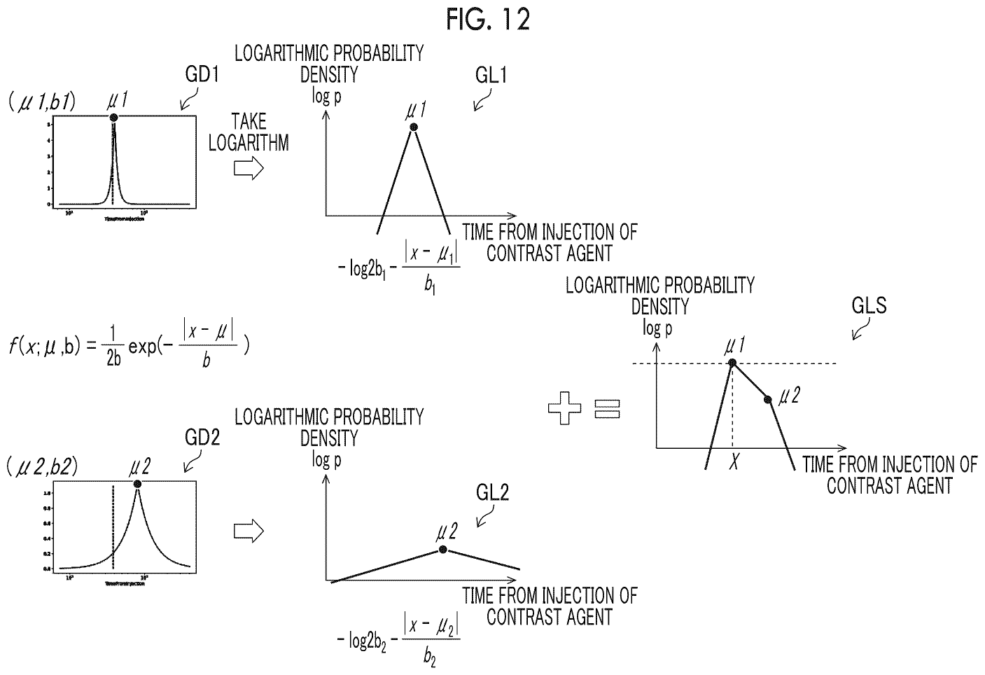

FIG. 12 is a diagram illustrating an example of the processes of the integration unit and the maximum point specification unit. Here, for simplicity of description, an example in which two number-of-seconds distributions estimated by the number-of-seconds distribution estimation unit 14 are integrated will be described. However, the same applies to a case where three or more number-of-seconds distributions are integrated.

A graph GD1 illustrated on the upper left side of FIG. 12 illustrates an example of a probability distribution P1 which is a number-of-seconds distribution represented by a parameter μ1 and a parameter b1 estimated for the input of a slice image IM1 by the number-of-seconds distribution estimation unit 14 illustrated in FIG. 8. In addition, the illustration of the slice image IM1 is omitted in FIG. 12. The slice image IM1 is illustrated in FIG. 16. The same applies to a slice image IM2 which will be described below.

The integration unit 16 takes a logarithm of the estimated number-of-seconds distribution to convert the number-of-seconds distribution into a logarithmic probability density and sums up a plurality of logarithmic probability densities to perform integration. This corresponds to calculating the product of the probabilities at the same number of seconds.

A graph GL1 illustrated on the upper middle side of FIG. 12 is an example of a logarithmic probability density logP1 obtained by taking a logarithm of the probability distribution P1. A graph GD2 illustrated on the lower left side of FIG. 12 is an example of a probability distribution P2 which is a number-of-seconds distribution represented by a parameter μ2 and a parameter b2 estimated for the input of a slice image IM2 by the number-of-seconds distribution estimation unit 14.

A graph GL2 illustrated on the lower middle side of FIG. 12 is an example of a logarithmic probability density logP2 obtained by taking a logarithm of the probability distribution P2. A graph GLS illustrated on the rightmost side of FIG. 12 is an example of a simultaneous logarithmic probability density obtained by integrating the logarithmic probability density logP1 and the logarithmic probability density logP2.

The maximum point specification unit 18 specifies a value x of the parameter u, at which the logarithmic probability is maximized, from the integrated logarithmic probability density. The process of the maximum point specification unit 18 can be represented by the following Expression 4.

$$\begin{matrix}x = {arg max}_{x} {\sum }_{i} ( - log 2 {b}_{i} - \frac{\vert x - {μ}_{i} \vert}{{b}_{i}} ) \\ = {arg max}_{x} {\sum }_{i} ( log {b}_{i} - \frac{\vert x - {μ}_{i} \vert}{{b}_{i}} ) \\ = {arg max}_{x} {\sum }_{i} \frac{\vert x - {μ}_{i} \vert}{{b}_{i}}\end{matrix} \qquad Expression 4$$

A portion after Σ, which is a target function of arg min illustrated on the right side of an equal sign described in the second row of Expression 4, corresponds to a loss function during training in machine learning which will be described below. In addition, the right side of the equal sign described in the third row corresponds to a weighted median expression. A parameter bi corresponding to the weight during integration dynamically changes according to the output of the regression estimation unit 22.

In the case of the integrated logarithmic probability density illustrated in the graph GLS of FIG. 12, the maximum point which is the input value at which the simultaneous logarithmic probability is maximized is the parameter μ1, and the parameter μ1 is selected as the final estimation result. In addition, the parameter μ1 is the estimation result of the slice image IM1 among the plurality of input slice images IMi. The integration unit 16 whose process is illustrated in FIG. 12 converts the number-of-seconds distribution into the logarithmic probability density and performs calculation. In short, the integration unit 16 performs a process of deriving a value, at which the simultaneous probability is maximized, as the final result, considering the simultaneous probability of probability distributions which are a plurality of number-of-seconds distributions estimated from a plurality of different inputs.

The Laplace distribution is adopted as the probability distribution model, and the simultaneous probability distribution which is the integrated distribution has the form of a weighted median. In a case where some of a plurality of estimation results are values that deviate significantly due to artifacts or the like, it is possible to suppress the influence of the outliers and to obtain an estimated value with high accuracy.

In addition, the parameter μ1 described in the embodiment is an example of the first parameter, and the parameter b1 is an example of the second parameter.

### Example 1 of Machine Learning Method

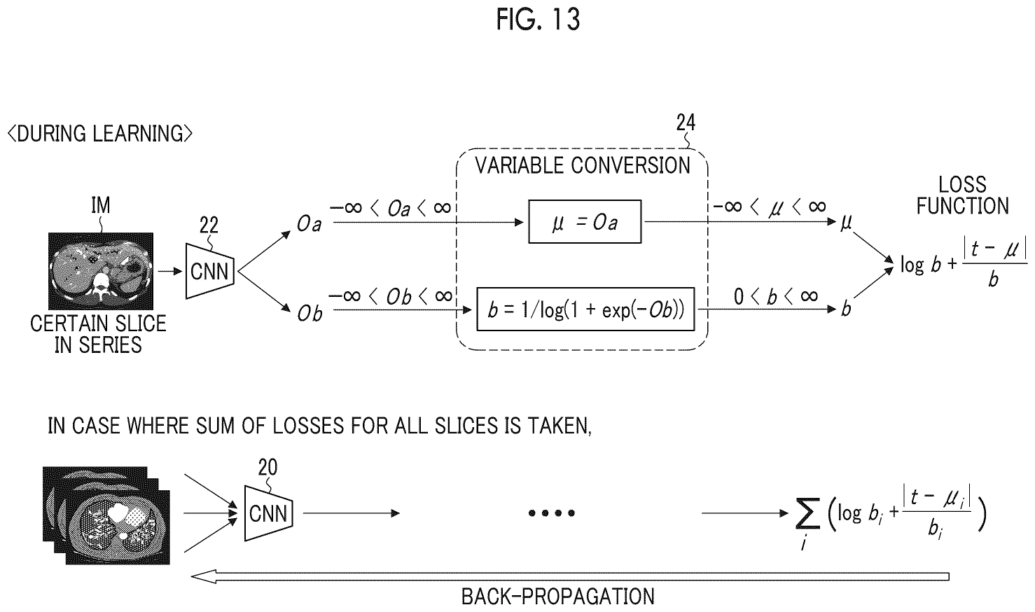

FIG. 13 is a diagram schematically illustrating an example of a machine learning method for generating a regression model used in the number-of-seconds distribution estimation unit. Training data used for machine learning includes the slice image IM as data for input and a teaching signal t as correct answer data corresponding to the input. The slice image IM may be a slice image constituting an image series of three-dimensional CT data, and the teaching signal t may be a value indicating ground truth which is the number of seconds from the injection of the contrast agent in a case where the image series to which the slice image IM belongs is captured.

For example, a plurality of training data items are generated by linking the corresponding teaching signals t to all of the slice images IM of the image series. The linking may be paraphrased as correspondence or association. The term “training” is synonymous with learning. The same teaching signal t may be linked with the slice images IM of the same image series. That is, the teaching signal t may be linked in units of image series.

For a plurality of image series, similarly, a plurality of training data items are generated by linking the corresponding teaching signals t to the slice images IM. An aggregate of the plurality of training data items generated in this way is used as a training data set.

A learning model 20 is configured using the CNN. The learning model 20 is used in combination with a variable conversion unit 24. In addition, the variable conversion unit 24 may be integrally incorporated into the learning model 20.

The slice image IM read out from the training data set is input to the learning model 20, and the learning model 20 outputs an estimated number-of-seconds value Oa and a score value Ob of the certainty of the estimated number-of-seconds value Oa. The variable conversion unit 24 performs variable conversion to convert the estimated number-of-seconds value Oa and the score value Ob into a parameter u and a parameter b of the probability distribution model.

A loss function L used during training is defined using Expression 5.

$$L = log b + \frac{\vert t - μ \vert}{b} \qquad Expression 5$$

As illustrated on the lower side of FIG. 13, in a case where the sum of the losses is taken for all of slices of the same image series, the sum of the losses is represented using Expression 6.

$${\sum }_{i} ( log {b}_{i} + \frac{\vert t - {μ}_{i} \vert}{b} ) \qquad Expression 6$$

A suffix i is an index for identifying each slice. A back-propagation method is applied using the sum of the losses represented by Expression 6, and the learning model 20 is trained using a stochastic gradient descent method in the same manner as in normal CNN training. In addition, the training of the learning model 20 is synonymous with updating the parameters of the learning model 20.

The learning model 20 is trained using a plurality of training data items including a plurality of image series such that the parameters of the learning model 20 are optimized to obtain a trained model. The trained model obtained in this way is used as the regression model of the number-of-seconds distribution estimation unit 14.

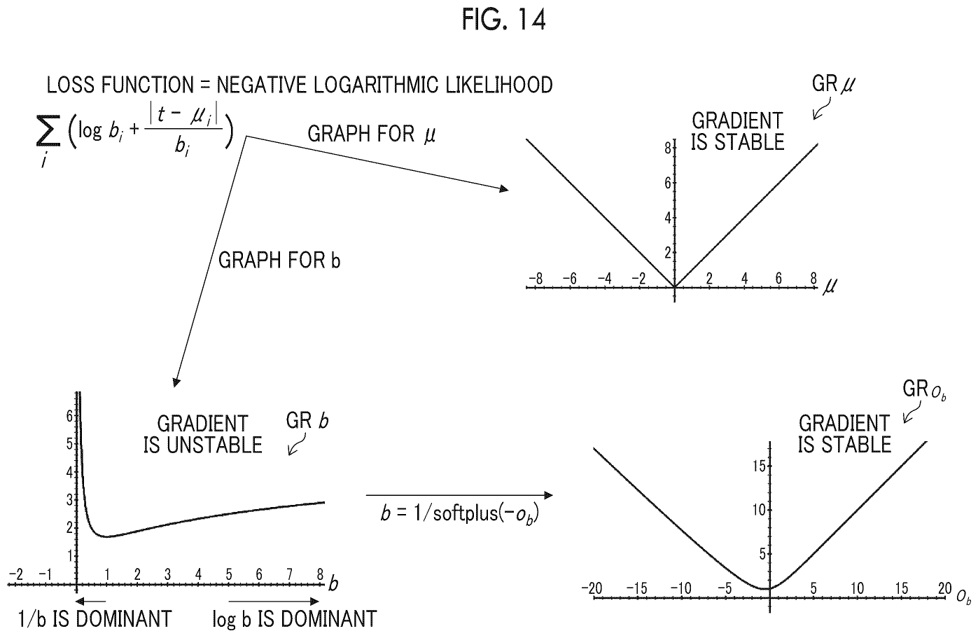

FIG. 14 is a diagram illustrating the loss function used during training. The loss function is negative logarithmic likelihood and directly optimizes an expression that is used for regression estimation using learning. The loss function maximizes the logarithmic likelihood of the teaching signal t at the number of seconds using learning. A graph for the parameter u of the loss function represented by Expression 5 is a graph GRμ illustrated in FIG. 14. In the graph GRμ, the gradient with respect to the parameter u is stable.

On the other hand, a graph for the parameter b of the loss function represented by Expression 5 is a graph GRb illustrated in FIG. 14. In the graph GRb, the gradient with respect to the parameter b is unstable. 1/b is dominant in a region in which the value of b is small, and logb is dominant in a region in which the value of b is large.

The graph GRb in which the gradient is unstable is converted into a graph GROb by performing variable conversion to convert the parameter b using a function such as b=1/softplus(−Ob). A softplus function is defined as softplus(x)=log(1+exp(x)). The function used for the variable conversion of the parameter b is a function that approaches −1/x at x→−∞ and approaches exp(x) at x→∞. The use of this function makes it possible to cancel the instability of the gradient.

### [Example of Hardware Configuration]

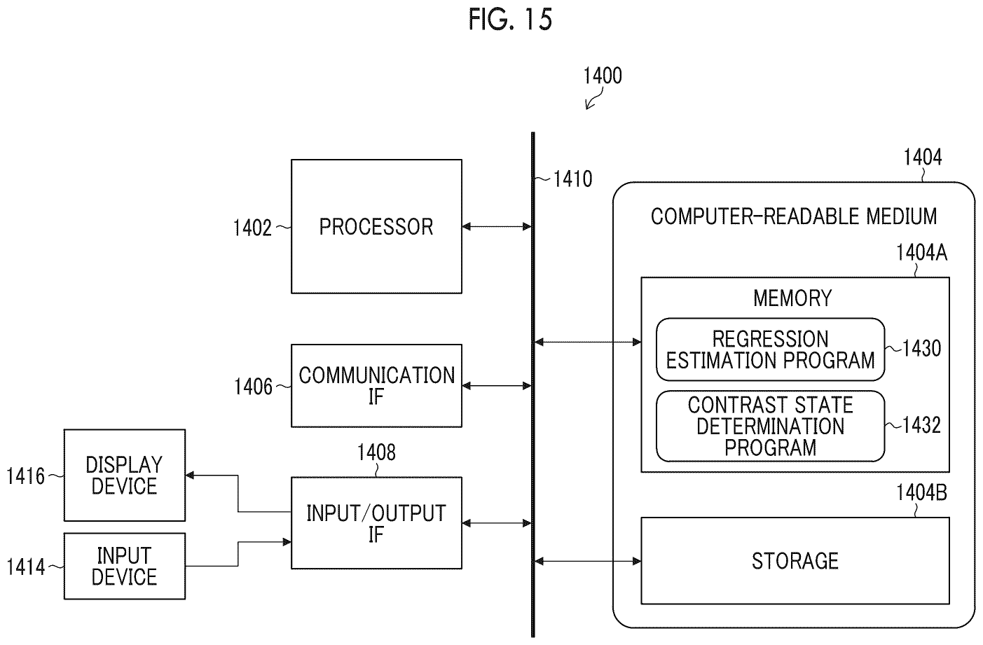

FIG. 15 is a block diagram schematically illustrating an example of a hardware configuration of the contrast state determination device according to the third embodiment. Here, components different from those of the contrast state determination device 1000 illustrated in FIG. 2 will be mainly described, and the description of components common to the contrast state determination device 1000 will be omitted as appropriate.

The contrast state determination device 1400 comprises a processor 1402, a computer-readable medium 1404, a communication interface 1406, an input/output interface 1408, and a bus 1410. The contrast state determination device 1400 may comprise an input device 1414 and a display device 1416. The computer-readable medium 1404 comprises a memory 1404A and a storage 1404B.

The computer-readable medium 1404 illustrated in FIG. 15 stores a regression estimation program 1430 instead of the number-of-seconds estimation program 1130 illustrated in FIG. 2. The processor 1402 executes one or more commands included in the regression estimation program 1430 to implement the functions of the regression estimation device 10 illustrated in FIG. 8. The regression estimation program 1430 can include a trained model.

The computer-readable medium 1404 stores a contrast state determination program 1432. The processor 1402 executes the contrast state determination program 1432 to implement a contrast state determination function of the contrast state determination device 1400.

### [Functional Configuration of Regression Estimation Device]

FIG. 16 is a functional block diagram illustrating an outline of processing functions of a regression estimation device used in the contrast state determination device according to the third embodiment. The processor 1402 of the contrast state determination device 1400 executes the regression estimation program 1430 stored in the memory 1404A to function as the data acquisition unit 12, the number-of-seconds distribution estimation unit 14, the integration unit 16, and the maximum point specification unit 18.

The data acquisition unit 12 acquires a plurality of slice images IMi. FIG. 16 illustrates an example in which n slice images IMi are acquired as in the example illustrated in FIG. 6.

The slice images IMi acquired through the data acquisition unit 12 are input to the regression estimation unit 22 of the number-of-seconds distribution estimation unit 14. The regression estimation unit 22 outputs a set of the estimated number-of-seconds value Oa and the score value Ob indicating the certainty of the estimated number-of-seconds value Oa from each of the input slice images IMi.

The variable conversion unit 24 converts the estimated number-of-seconds value Oa output from the regression estimation unit 22 into a parameter μi of the probability distribution model. The variable conversion unit 24 converts the score value Ob of the certainty output from the regression estimation unit 22 into a parameter bi of the probability distribution model. A probability distribution Pi of the number of seconds is estimated on the basis of the parameter μi and the parameter bi.

In the regression estimation device 10, a plurality of slice images IMi included in the same image series are input, a set of the estimated number-of-seconds value Oa and the score value Ob is estimated for each of the slice image IMi and converted into a set of the parameters μi and bi. Then, the probability distribution Pi of the number of seconds is estimated.

The integration unit 16 performs a process of integrating a plurality of probability distributions Pi obtained on the basis of the input of the plurality of slice images IMi. In the example illustrated in FIG. 16, a logarithmic conversion unit 26 takes a logarithm of the probability distribution Pi to convert the probability distribution Pi into a logarithmic probability density logPi, and an integrated distribution generation unit 28 calculates the sum of the logarithmic probability densities logPi to obtain an integrated distribution.

The maximum point specification unit 18 specifies the maximum point, which is the value of the number of seconds at which the probability is maximized, from the integrated distribution and outputs the specified value of the number of seconds as a final estimated value. In addition, the maximum point specification unit 18 may be incorporated into the integration unit 16.

### Operation and Effect of Third Embodiment

The contrast state determination device 1400 according to the third embodiment can obtain the following operation and effect.

[1]

The following configuration can be used: the probability distribution Pi having the estimated number-of-seconds value Oa as a random variable is estimated on the basis of the estimated number-of-seconds value Oa and the score value Ob indicating the certainty of the estimated number-of-seconds value Oa; and a value at which the product of the probabilities at the same random variable is maximized is specified on the basis of each probability distributions Pi in a plurality of sets.

[2]

The value at which the simultaneous probability is maximized can be calculated, on the basis of a plurality of probability distributions Pi estimated from the input of a plurality of slice images IMi, to derive the estimated number-of-seconds value Oa with high accuracy in consideration of the score value Ob indicating the certainty estimated according to the input.

[3]

An expression used for inference of the regression model is directly optimized using machine learning.

[4]

Since the Laplace distribution is adopted as the probability distribution model, learning is stable and is robust to label noise to some extent. Further, the simultaneous probability distribution has the form of a weighted median. In a case where one of the estimation results for some of the inputs deviates significantly due to artifacts or the like, the learning is less likely to be affected by the outlier and is further robust. Furthermore, it is possible to extract the image used for estimating the final estimated value, which is the final result, from a plurality of images used for the input.

### Contrast State Determination Device According to Fourth Embodiment

In the third embodiment, the Laplace distribution is used as the probability distribution model of the number-of-seconds distribution. However, the present invention is not limited thereto, and other probability distribution models may be applied. In the fourth embodiment, an example will be described in which a Gaussian distribution is used instead of the Laplace distribution.

A hardware configuration of a contrast state determination device according to the fourth embodiment may be the same as that of the contrast state determination device 1400 according to the third embodiment. Here, components different from those of the contrast state determination device 1400 according to the third embodiment will be mainly described, and components common to the contrast state determination device 1400 will be omitted as appropriate. In the contrast state determination device according to the fourth embodiment, the content of the processes of the processing units of the number-of-seconds distribution estimation unit 14, the integration unit 16, and the maximum point specification unit 18 is different from that in the third embodiment.

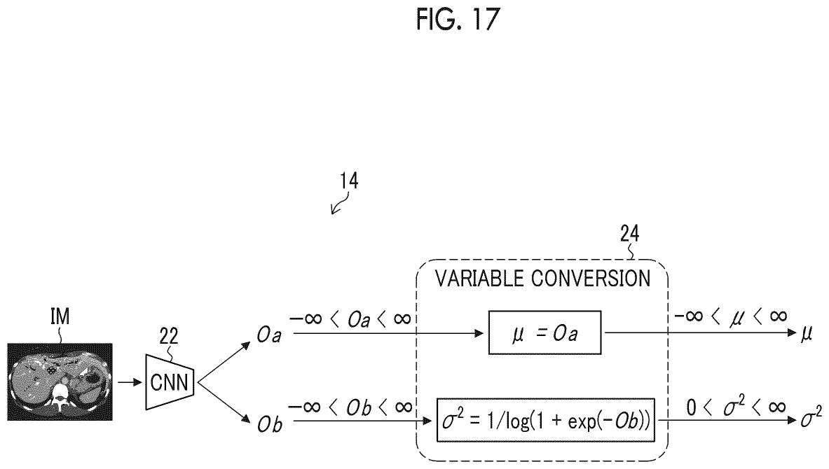

FIG. 17 is a diagram illustrating Example 2 of the process of the number-of-seconds distribution estimation unit of the regression estimation device provided in the contrast state determination device according to the fourth embodiment. In the contrast state determination device according to the fourth embodiment, the process illustrated in FIG. 17 is applied instead of the process illustrated in FIG. 9.

The variable conversion unit 24 according to the fourth embodiment converts the score value Ob of the certainty into a parameter σ² using Expression 7 instead of Expression 2.

σ²=1/log(1+exp(−Ob)) Expression 7

σ² plays a role of certainty. σ² corresponds to a dispersion, and σ corresponds to a standard deviation.

The Gaussian distribution is represented using a function represented by Expression 8.

$$f ( x ; μ , σ ) = \frac{1}{\sqrt{2 π {o}^{2}}} exp ( - \frac{{( x - μ )}^{2}}{2 {σ}^{2}} ) \qquad Expression 8$$

The reason for converting the score value Ob into σ² which is a positive value is the same as that in the third embodiment. In a case where the parameter σ² is a negative value, the Gaussian distribution is not established as the probability distribution, and it is necessary to ensure that the parameter σ² is a positive value (σ²>0).

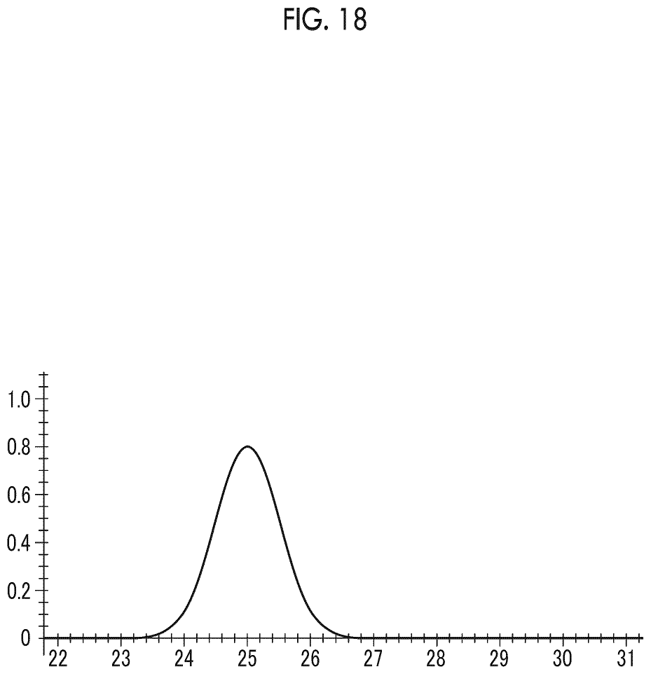

FIG. 18 is a graph of the number-of-seconds distribution that is estimated on the basis of the parameter u and the parameter σ² estimated by the number-of-seconds distribution estimation unit. FIG. 18 illustrates an example of a graph of the number-of-seconds distribution.

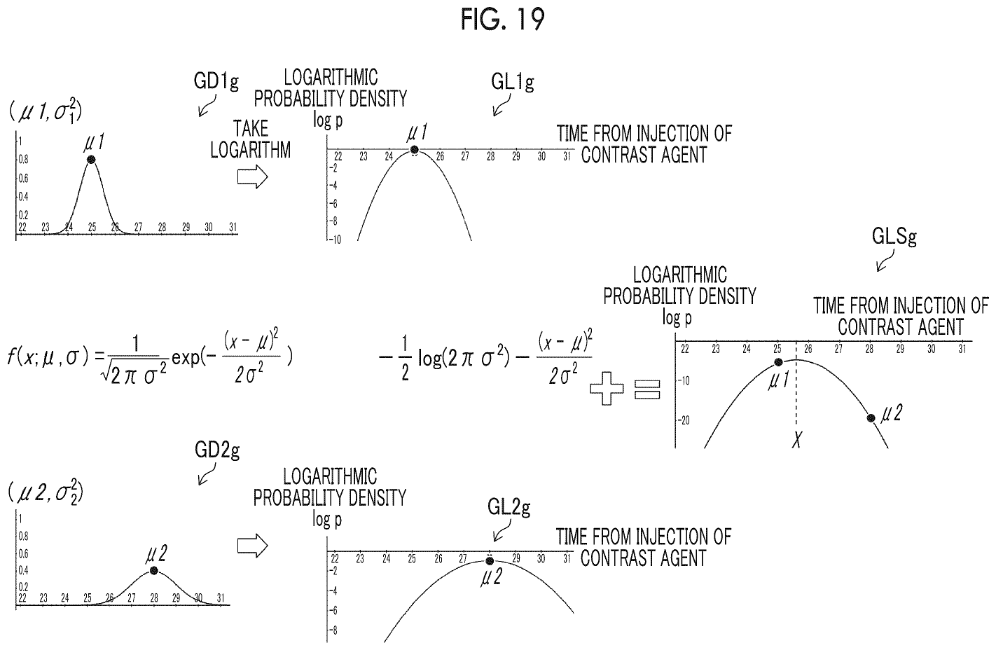

FIG. 19 is a diagram illustrating an example of processes of the integration unit and the maximum point specification unit of the regression estimation device provided in the contrast state determination device according to the fourth embodiment. Here, an example in which two number-of-seconds distributions estimated by the number-of-seconds distribution estimation unit 14 are integrated will be described.

A graph GD1g illustrated on the upper left side of FIG. 19 is an example of a probability distribution P1 which is a number-of-seconds distribution represented by a parameter μ1 and a parameter σ²₁ estimated by the number-of-seconds distribution estimation unit 14 illustrated in FIG. 17. The integration unit 16 takes a logarithm of the estimated number-of-seconds distribution to convert the number-of-seconds distribution into a logarithmic probability density and calculates the sum of a plurality of logarithmic probability densities to perform integration. This corresponds to calculating the product of the probabilities at the same number of seconds.

A graph GL1g illustrated on the upper middle side of FIG. 19 is an example of a logarithmic probability density logP1 obtained by taking a logarithm of the probability distribution P1. A graph GD2g illustrated on the lower left side of FIG. 19 is an example of a probability distribution P2 which is a number-of-seconds distribution represented by a parameter μ2 and a parameter σ²₂ estimated by the number-of-seconds distribution estimation unit 14. A graph GL2g illustrated on the lower middle side of FIG. 19 is an example of a logarithmic probability density logP2 obtained by taking a logarithm of the probability distribution P2.

A graph GLSg illustrated on the rightmost side of FIG. 19 is an example of a simultaneous logarithmic probability density obtained by integrating the logarithmic probability density logP1 and the logarithmic probability density logP2.

The maximum point specification unit 18 specifies a value x, at which the logarithmic probability is maximized, from the integrated simultaneous logarithmic probability density. The process of the maximum point specification unit 18 is represented by Expression 9.

$$\begin{matrix}x = {arg max}_{x} {\sum }_{i} ( - log 2 π {σ}_{i}^{2} - \frac{{( x - {μ}_{i} )}^{2}}{2 {σ}^{2}} ) \\ = {arg max}_{x} {\sum }_{i} ( log {σ}_{i}^{2} - \frac{{( x - {μ}_{i} )}^{2}}{2 {σ}^{2}} ) \\ = {arg max}_{x} {\sum }_{i} \frac{{( x - {μ}_{i} )}^{2}}{{σ}^{2}}\end{matrix} \qquad Expression 9$$

A portion after Σ, which is a target function of arg min illustrated on the right side of an equal sign described in the second row of Expression 9, corresponds to a loss function during training in machine learning which will be described below. In addition, the right side of the equal sign described in the third row corresponds to a weighted average expression.

In the case of the integrated logarithmic probability density in the graph GLSg illustrated in FIG. 19, the value x indicating the maximum point which is the input value at which the logarithmic probability is maximized is selected as the final result which is the final estimation result.

### Example 2 of Machine Learning Method

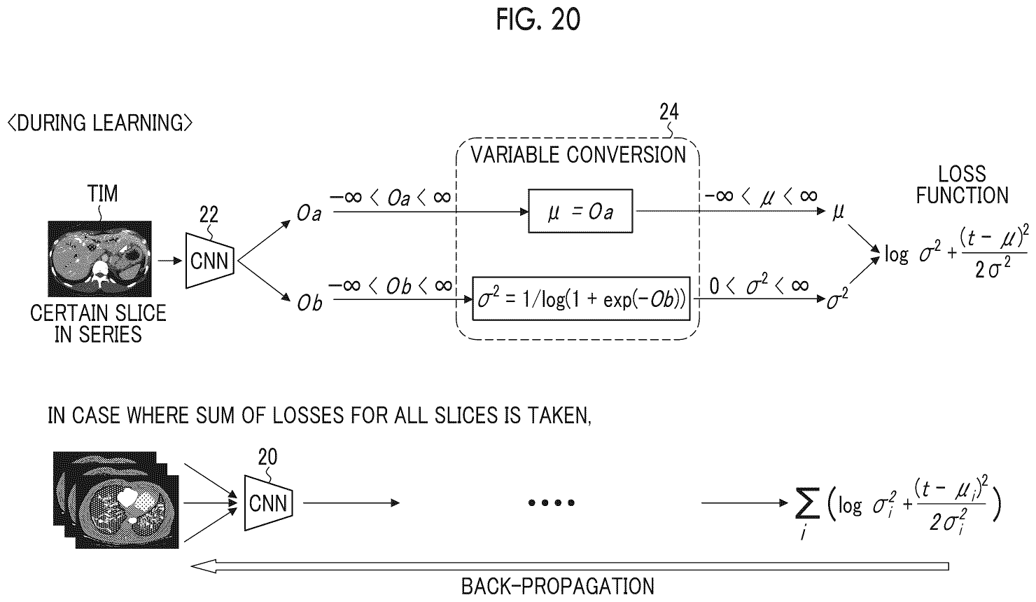

FIG. 20 is a diagram schematically illustrating an example of a machine learning method for generating a regression model used in the number-of-seconds distribution estimation unit provided in the contrast state determination device according to the fourth embodiment. Training data used in learning may be the same as that in the third embodiment. Differences from Example 1 illustrated in FIG. 13 will be mainly described with reference to FIG. 20.

In a case where a slice image TIM read out from a training data set is input to the learning model 20, the learning model 20 outputs an estimated number-of-seconds value Oa and a score value Ob of the certainty of the estimated number-of-seconds value Oa. The variable conversion unit 24 performs variable conversion to convert the estimated number-of-seconds value Oa and the score value Ob of the certainty into a parameter u and a parameter σ² of the probability distribution model, respectively.

A loss function L during training is defined using Expression 10.

$$L = log {σ}^{2} + \frac{{( t - μ )}^{2}}{2 {σ}^{2}} \qquad Expression 10$$

As illustrated on the lower side of FIG. 20, in a case where the sum of losses is taken for all of slices of the same image series, the sum is represented by Expression 11.

$${\sum }_{i} ( log {σ}_{i}^{2} + \frac{{( t - {μ}_{i} )}^{2}}{2 {σ}_{i}^{2}} ) \qquad Expression 11$$

The back-propagation method is applied using the sum of the losses represented by Expression 11, and the learning model 20 is trained using the stochastic gradient descent method in the same manner as in normal CNN training. The learning model 20 is trained using a plurality of training data items including a plurality of image series such that the parameters of the learning model 20 are optimized to obtain a trained model. The trained model obtained in this way is applied to the number-of-seconds distribution estimation unit 14.

### Operation and Effect of Fourth Embodiment

The contrast state determination device according to the fourth embodiment can obtain the same operation and effect as the contrast state determination device 1000 according to the first embodiment, the contrast state determination device 1200 according to the second embodiment, and the contrast state determination device 1400 according to the third embodiment.

### Modification Example 1

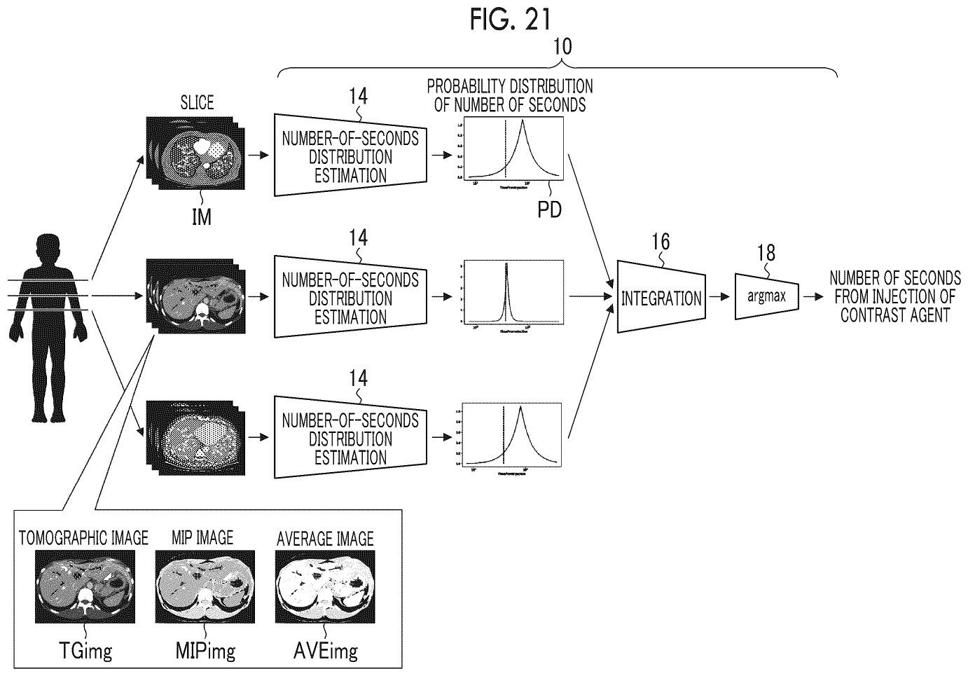

FIG. 21 is a diagram illustrating Modification Example 1 of the image used for input to the number-of-seconds estimation device. In each of the first to fourth embodiments, the slice images IM obtained by dividing three-dimensional CT data into slices at equal intervals are used as the input. However, the image to be processed is not limited thereto. For example, as illustrated in FIG. 21, instead of the tomographic images TGimg, MIP images MIPimg configured at equal intervals, an average image AVEimg generated from a plurality of slice images, or the like may be used. In addition, MIP is an abbreviation of Maximum Intensity Projection.

Further, the image used for input is not limited to the two-dimensional image and may be a three-dimensional image. For example, a three-dimensional partial image may be used as the input. Furthermore, in a case where a plurality of slice images MIi are used, three-dimensional partial images at different positions included in the same image series may be input. In addition, the MIP image MIPimg and the average image AVEimg described in the embodiment are examples of generated images that are generated on the basis of the partial images included in the three-dimensional image.

### Modification Example 2

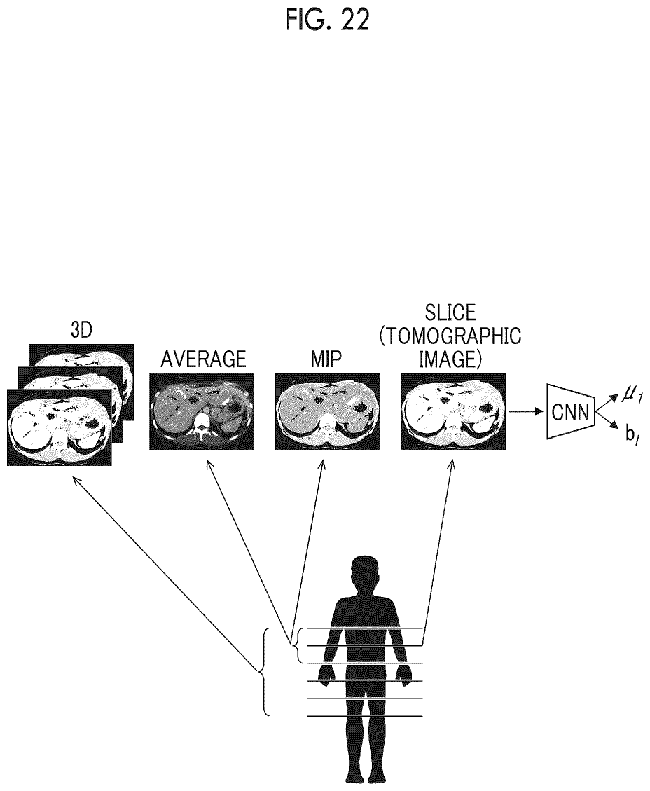

FIG. 22 is a diagram illustrating Modification Example 2 of the image used for input to the number-of-seconds estimation device. The input to the number-of-seconds distribution estimation unit 14 illustrated in FIG. 8 and the like may be a combination of a plurality of types of data elements. For example, as illustrated in FIG. 22, at least one of the three-dimensional image, the slice image, the MIP image, or the average image which is a partial image of CT data of the same image series can be used as the input. A combination of the plurality of types of images may be input to the number-of-seconds distribution estimation unit 14 to obtain an output of an estimated number-of-seconds value and the certainty thereof. The three-dimensional image referred to here means a set of a plurality of slice images.

For example, a combination of the average image and the MIP image may be input to the number-of-seconds distribution estimation unit 14 to estimate the number-of-seconds distribution. In addition, 3D illustrated in FIG. 22 indicates the three-dimensional image.

### [Example of Configuration of Medical Imaging System]

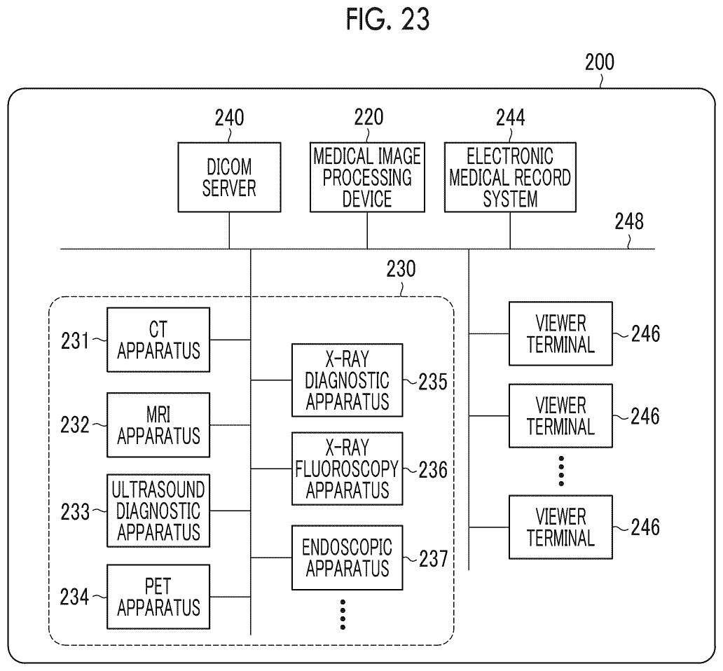

FIG. 23 is a block diagram illustrating an example of a configuration of a medical information system in which the contrast state determination device is used. The contrast state determination device 1000 and the like described in the first to fourth embodiments can be incorporated into a medical image processing device 220 illustrated in FIG. 23.

A medical information system 200 is a computer network constructed in a medical institution such as a hospital. The medical information system 200 comprises a modality 230 that captures a medical image, a DICOM server 240, the medical image processing device 220, an electronic medical record system 244, and a viewer terminal 246. Elements of the medical information system 200 are connected through a communication line 248. The communication line 248 may be a local communication line in the medical institution. Further, a portion of the communication line 248 may be a wide area communication line.

Specific examples of the modality 230 include a CT apparatus 231, an MRI apparatus 232, an ultrasound diagnostic apparatus 233, a PET apparatus 234, an X-ray diagnostic apparatus 235, an X-ray fluoroscopy apparatus 236, and an endoscopic apparatus 237. There may be various combinations of types of the modalities 230 connected to the communication line 248 for each medical institution. In addition, MRI is an abbreviation of Magnetic Resonance Imaging. PET is an abbreviation of Positron Emission Tomography.

The DICOM server 240 is a server that operates according to the specifications of DICOM. The DICOM server 240 is a computer that stores various types of data including the images captured by the modality 230 and that manages various types of data. The DICOM server 240 comprises a large-capacity external storage device and a database management program.

The DICOM server 240 communicates with other devices through the communication line 248 to transmit and receive various types of data including image data. The DICOM server 240 receives the image data generated by the modality 230 and other various types of data through the communication line 248, stores the data in a recording medium, such as a large-capacity external storage device, and manages the data. In addition, the storage format of the image data and the communication between the devices via the communication line 248 are based on a DICOM protocol.

The medical image processing device 220 can acquire data from the DICOM server 240 or the like via the communication line 248. The medical image processing device 220 performs image analysis and various other processes on the medical image captured by the modality 230. The medical image processing device 220 may be configured to perform various computer-aided diagnosis analysis processes, such as a process of recognizing a lesion region and the like from an image, a process of specifying a classification, such as a disease name, and a segmentation process of recognizing a region, such as an organ, in addition to the processing functions of the regression estimation device 10. In addition, the computer-aided diagnosis can be referred to as CAD which is an abbreviation of Computer Aided Diagnosis or Computer Aided Detection.

Further, the medical image processing device 220 can transmit a processing result to the DICOM server 240 and the viewer terminal 246. Furthermore, the processing functions of the medical image processing device 220 may be provided in the DICOM server 240 or the viewer terminal 246.

Various types of data stored in the database of the DICOM server 240 and various types of information including the processing result generated by the medical image processing device 220 can be displayed on the viewer terminal 246.

The viewer terminal 246 is an image viewing terminal called a PACS viewer or a DICOM viewer. A plurality of viewer terminals 246 may be connected to the communication line 248. In addition, PACS is an abbreviation of Picture Archiving and Communication System. The form of the viewer terminal 246 is not particularly limited and may be, for example, a personal computer, a workstation, or a tablet terminal.

### [For Program for Operating Computer]

A program that causes a computer to implement the processing functions of the contrast state determination device 1000 and the like can be recorded on a computer-readable medium which is a non-transitory tangible information storage medium, such as an optical disk, a magnetic disk, or a semiconductor memory. Then, the program can be provided through the information storage medium.

In addition, instead of the aspect in which the program is stored in the non-transitory tangible computer-readable medium and then provided, program signals may be provided as a download service using a telecommunication line such as the Internet.

Further, some or all of the processing functions of the contrast state determination device 1000 and the like may be implemented by cloud computing or may be provided as a SasS service. In addition, SasS is an abbreviation of Software as a Service.

### [For Hardware Configuration of Each Processing Unit]

A hardware structure of processing units performing various processes, such as the data acquisition unit 12, the number-of-seconds distribution estimation unit 14, the integration unit 16, the maximum point specification unit 18, the regression estimation unit 22, the variable conversion unit 24, the logarithmic conversion unit 26, the integrated distribution generation unit 28, in the contrast state determination device 1000 and the like is the following various processors.

The various processors include, for example, a CPU which is a general-purpose processor executing a program to function as various processing units, a GPU which is a processor specialized for image processing, a programmable logic device, such as a field programmable gate array (FPGA), which is a processor whose circuit configuration can be changed after manufacture, and a dedicated electric circuit, such as an ASIC, which is a processor having a dedicated circuit configuration designed to perform a specific process.

In addition, the programmable logic device can be referred to as a PLD which is an abbreviation of Programmable Logic Device in English. ASIC is an abbreviation of Application Specific Integrated Circuit.

One processing unit may be configured by one of these various processors or may be configured by two or more processors of the same type or different types. For example, one processing unit may be configured using a plurality of FPGAs, a combination of a CPU and an FPGA, or a combination of a CPU and a GPU.

Further, a plurality of processing units may be configured by one processor. A first example of the configuration in which a plurality of processing units are configured by one processor is an aspect in which one processor is configured by a combination of one or more CPUs and software and functions as a plurality of processing units. A representative example of this aspect is a client computer or a server computer. A second example of the configuration is an aspect in which a processor that implements the functions of the entire system including a plurality of processing units using one IC chip is used. A representative example of this aspect is a system on chip. In addition, the system on chip can be referred to a SoC which is an abbreviation of System On a Chip. IC is an abbreviation of Integrated Circuit.

As described above, the various processing units are configured using one or more of the various processors as the hardware structure. In addition, specifically, the hardware structure of the various processors is an electric circuit (circuitry) obtained by combining circuit elements such as semiconductor elements.

The technical scope of the present invention is not limited to the scope described in the embodiments. The configurations and the like in each embodiment can be appropriately combined between the embodiments without departing from the gist of the present invention.

### EXPLANATION OF REFERENCES

10: regression estimation device
12: data acquisition unit
14: number-of-seconds distribution estimation unit
16: integration unit
18: maximum point specification unit
20: learning model
22: regression estimation unit
24: variable conversion unit
26: logarithmic conversion unit
28: integrated distribution generation unit
200: medical information system
220: medical image processing device
230: modality
231: CT apparatus
232: MRI apparatus
233: Ultrasound diagnostic apparatus
234: PET apparatus
235: X-ray diagnostic apparatus
236: X-ray fluoroscopy apparatus
237: endoscopic apparatus
240: DICOM server
244: electronic medical record system
246: viewer terminal
248: communication line
1000: contrast state determination device
1002: image receiving unit
1004: number-of-seconds estimation unit
1006: contrast state determination unit
1008: output unit
1010: conversion table
1102: processor
1104: computer-readable medium
1104A: memory
1104B: storage
1106: communication interface
1108: input/output interface
1110: bus
1114: input device
1116: display device
1130: number-of-seconds estimation program
1132: contrast state determination program
1200: contrast state determination device
1202: image receiving unit
1204: number-of-seconds estimation unit
1206: contrast state determination unit
1208: output unit
1210: conversion table
GD1: graph
GD1g: graph
GD2: graph
GD2g: graph
GL1: graph
GL1g: graph
GL2: graph
GL2g: graph
GLS: graph
GLSg: graph
GRb: graph
GRμ: graph
GROb: graph
IM: slice image
IM1: slice image
IM2: slice image
IMn: slice image
IMi: slice image
TIM: slice image
Oa: estimated number-of-seconds value
Ob: score value
P1: probability distribution
P2: probability distribution
Pi: probability distribution
PD: number-of-seconds distribution
S10 to S18. S100 to S108: each step of contrast state determination method

What is claimed is:

1. A medical image processing device comprising:
one or more processors; and
one or more memories that store a program to be executed by the one or more processors,
wherein the one or more processors execute commands of the program to receive an input of an image generated by performing contrast imaging and to estimate an elapsed period from start of injection of a contrast agent in the image on the basis of image analysis of the image.

2. The medical image processing device according to claim 1,
wherein the one or more processors determine a contrast state of the image on the basis of the estimated elapsed period.

3. The medical image processing device according to claim 1,
wherein the one or more processors receive an input of at least one of a slice image, a partial image included in a three-dimensional image, a generated image generated on the basis of the partial image included in the three-dimensional image, or the three-dimensional image as the image.

4. The medical image processing device according to claim 1,
wherein the one or more processors estimate the elapsed period using a trained regression model.

5. The medical image processing device according to claim 1,
wherein the one or more processors
receive an input of a first image,
receive an input of a second image that belongs to the same image series as the first image and that is captured at a position different from that of the first image,
estimate a first elapsed period which is an elapsed period from the start of the injection of the contrast agent in the first image,
estimate a second elapsed period which is an elapsed period from the start of the injection of the contrast agent in the second image, and
estimate the elapsed period belonging to the image series on the basis of the first elapsed period and the second elapsed period.

6. The medical image processing device according to claim 5,
wherein the one or more processors integrate the first elapsed period and the second elapsed period to estimate the elapsed period.

7. The medical image processing device according to claim 5,
wherein the one or more processors estimate the first elapsed period and the second elapsed period using a trained regression model.

8. The medical image processing device according to claim 7,
wherein the one or more processors
estimate an estimated value output from the regression model and a certainty of the output estimated value for each of the first image and the second image, and
integrate estimation results for each of the first image and the second image on the basis of the estimated value and the certainty estimated for each of the first image and the second image using the regression model.

9. The medical image processing device according to claim 8,
wherein the one or more processors
estimate a probability distribution having the estimated value as a random variable for each of the first image and the second image on the basis of the estimated value and the certainty of the estimated value,
integrate the probability distributions of the first image and the second image to generate an integrated distribution, and
specify a final estimated value on the basis of the integrated distribution.

10. The medical image processing device according to claim 9,
wherein the one or more processors
perform variable conversion to convert the estimated value output from the regression model into a first parameter of a probability distribution model, and
perform variable conversion to convert a value indicating the certainty output from the regression model into a second parameter of the probability distribution model.

11. The medical image processing device according to claim 10,
wherein the probability distribution model is a Laplace distribution.

12. The medical image processing device according to claim 10,
wherein the probability distribution model is a Gaussian distribution.

13. The medical image processing device according to claim 9,
wherein the one or more processors
perform logarithmic conversion to take a logarithm of the probability distribution,
calculate a sum of logarithmic probability densities corresponding to the probability distributions of the first image and the second image during the integration, and
calculate a value at which a simultaneous logarithmic probability density is maximized.

14. The medical image processing device according to claim 8,
wherein the regression model includes a trained model generated by performing machine learning using training data in which an image for input and a teaching signal are associated with each other.

15. The medical image processing device according to claim 8,
wherein the regression model is configured using a convolutional neural network.

16. The medical image processing device according to claim 5,
wherein the first image and the second image include different partial images included in a three-dimensional image.

17. The medical image processing device according to claim 5,
wherein the first image and the second image include generated images that are generated on the basis of different partial images included in a three-dimensional image.

18. The medical image processing device according to claim 5,
wherein the first image and the second image include three-dimensional images.

19. A medical image processing method comprising:
causing a computer to receive an input of an image generated by performing contrast imaging and to estimate an elapsed period from start of injection of a contrast agent in the image on the basis of image analysis of the image.

20. A non-transitory, computer-readable tangible recording medium on which a program for causing, when read by a computer, the computer to execute the medical image processing method according to claim 19 is recorded.
